(19) United States
(12) Patent Application Publication
(10) Pub. No.: US 2019/0117121 A1
(43) Pub. Date: Apr. 25, 2019

(54) System and Methods for Gait and Running Functional Improvement and Performance Training

(71) Applicant: University of Pittsburgh - Of the Commonwealth System of Higher Education, Pittsburgh, PA (US)

(72) Inventors: Krista Lee Kutina, Pittsburgh, PA (US); Goeran Fiedler, Pittsburgh, PA (US)

(21) Appl. No.: 16/106,298

(22) Filed: Aug. 21, 2018

Related U.S. Application Data
(60) Provisional application No. 62/576,339, filed on Oct. 24, 2017.

(51) Int. Cl. A61B 5/11; A61B 5/00

(52) U.S. Cl. CPC A61B 5/112; A61B 5/1038; A61B 5/486

(57) ABSTRACT

A gait analysis and training system is provided, as well as methods of training, e.g., retraining gait in a patient. The system provides real-time external feedback to a patient is portable so that use is not limited to a clinic.

### CROSS REFERENCE TO RELATED APPLICATIONS

This application claims the benefit of U.S. Provisional Patent Application No. 62/576,339, filed Oct. 24, 2017, which is incorporated herein by reference in its entirety.

The present disclosure relates to devices, systems, and methods for gait and running analysis and training of an individual based on sensed information relayed in real time in a wearable fashion.

Gait dysfunction is an impairment that can effect multiple patient populations, both neurologic and orthopedic, and become chronic and linger for years. There are subsets of the patient population that are particularly vulnerable to problematic and chronic gait dysfunction such as Parkinson's disease, stroke, osteoarthritis (OA), and limb loss. In 2007 patients with limb loss compiled approximately 1.7 million people and it has been estimated that by 2050, this rate is expected to double to 3.6 million in the United States. This is of clinical concern in that forced compensations from the loss of sensory feedback, neuromuscular control, and pain that affect forward propulsion and weight acceptance throughout the gait cycle, could have the consequence of destructive secondary joint issues and increased energy cost. In addition, lack of plantar flexion and normal ankle motion are linked to most amputee gait deviations, including asymmetrical gait timing. Other typical deviations include trunk shifts which can result in low back problems as well as increased misdirected loads through the ankle, knee and hip of both the surgical and intact limbs putting the patient at higher risk of cartilage degradation and secondary complications of arthritis. Not only can the kinematic variables that are a part of these dysfunctional lower extremity movement patterns be retrained, but they also can be retained. Gait retraining as an intervention has demonstrated promise in many populations, including the amputee population. This could lead to improved lower extremity function, which leads to improved energy consumption and to extended prosthesis life as well. Symmetry also has been an issue in amputee gait, and, as analysis has evolved from only qualitative to quantitative measurements of temporal, kinetic and kinematic, and combinations of all, the most prominent asymmetries have been determined as shortened stance times and decreased ground reaction forces.

Additional populations are at risk for gait or lower extremity loading issues if biomechanical discrepancies are present. Once chronic compensations begin, it is not hard to imagine how this triggers a cascade of secondary musculoskeletal issues. These compensations can become stubborn ingrained patterns. These chronic compensations then cause degradation of secondary joint tissues and can increase the already staggering 50% risk of osteoarthritis (OA) that we have in our lifetime. These secondary complications can be costly, cause increased energy costs, cause secondary injuries, and loss of function and quality of life.

When the pathological gait pattern has become habitual, gait retraining and associated physical therapy can help mitigate the adverse effects. A persisting problem with this objective has been the lack of effective methods that promote motor learning and retention. The traditional approach is to use demonstration, verbal cues, targeted strengthening in a non-dynamic manner. This includes providing instructions to the patients on how to change their motion patterns and is limited by the time constraints that come with scheduled appointments, as well as by the nature of the feedback, which almost inevitably focuses the attention of the patient internally (e.g., by giving instructions on how to move an extremity or load the limb with landing cues).

Methods and devices useful for training or retraining a gait or running pattern are desirable, not only to avoid further improper compensations that can cause secondary injuries or degradation to other orthopedic structures, but to improve recovery times, and improve prevention strategies.

### SUMMARY

In one aspect, a gait analysis, training, and retraining system comprising: a sensor configured to measure one or more attributes of gait of a patient; and a controller in communication with a sensory output device, the controller configured to, repeatedly, monitor a patient's gait in real-time and to provide real-time feedback to the patient receive and process information from the sensor representative of one or more attributes of the gait of the patient; generate a data set corresponding to the information from the sensor representative of one or more attributes of the gait of the patient; compare the generated data set to reference data indicating optimal values for a data set corresponding to the information from the sensor representative of one or more attributes of the gait of a patient; and cause the display to provide feedback comprising gait analysis based, at least in part, on the comparison between the generated data set and the reference data and at least indicating in the feedback if the generated data set is within defined tolerances relative to the reference data.

In another aspect, a method of analyzing and/or training gait in a patient, the method comprising, using a computer-implemented process, repeatedly, receiving and processing, in a computer, information from a sensor on the patient configured to measure one or more attributes of gait of a patient representative of one or more attributes of the gait of the patient during one or more physical actions relating to gait, performed by the patient; generating, in the computer, a data set corresponding to the information from the sensor representative of one or more attributes of the gait of the patient; comparing, in the computer, the generated data set to reference data indicating optimal values for a data set corresponding to the information from the sensor representative of one or more attributes of the gait of a patient; and generating, with the computer, an output causing a sensory output device to provide feedback comprising gait analysis based, at least in part, on the comparison between the generated data set and the reference data and at least indicating in the feedback if the generated data set is within defined tolerances relative to the reference data.

### BRIEF DESCRIPTION OF THE DRAWINGS

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limit of the invention.

FIG. 1A is a schematic drawing of an exemplary wearable sensor device including an inertial measurement unit according to an aspect of the disclosure; FIG. 1B is a schematic drawing of internal circuitry of one version of the exemplary wearable sensor device of FIG. 1A; FIG. 1C is a schematic drawing of internal circuitry of another version of the exemplary wearable sensor device of FIG. 1A;

FIG. 2 is a schematic drawing of a trans-tibial prosthesis with an integrated sensor.

FIG. 3 is a schematic drawing of a movement analysis system including the wearable sensor device of FIG. 1A;

FIG. 4 is a flow chart of an exemplary process for data collection from a wearable sensor device with an F/T sensor, or inertial measurement unit, according to an aspect of the disclosure.

FIG. 5: Exemplary architectural framework of preliminary real time system to be tested. The described colors correspond to Red (Percent Stance Phase calculation<58%, Green “ideal, Percent Phase Calculation between 58% and 63%, and Orange “overcompensation” corresponds to percent Stance Phase calculations>63% however a cut off is set to 80%.

FIG. 6 provides graphs showing an exemplary data set obtained from and/or calculated from an F/T sensor in prosthesis (trans-tibial amputation) including Fx, Fy, Fz values, distal Mx (dMx, at ankle), proximal My (pMy, at knee), and proximal Mz (pMz, at knee).

FIG. 7 is a flow chart of a process for analyzing a data set obtained from an F/T sensor, or inertial measurement unit, according to an aspect of the disclosure.

FIG. 8 provides real time ground reaction force feedback as relayed from i-Pecs™ sensor.

FIG. 9 is a schematic of an integrated wearable feedback system.

FIG. 10: Correlation between stance/step ratio (the feedback variable) and overall gait symmetry.

### DETAILED DESCRIPTION OF THE INVENTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values.

As used herein, the singular form of “a”, “an”, and “the” include plural referents unless the context clearly dictates otherwise.

As used herein, the terms “right”, “left”, “top”, “bottom”, and derivatives thereof shall relate to the invention as it is oriented in the drawing figures. However, it is to be understood that the invention can assume various alternative orientations and, accordingly, such terms are not to be considered as limiting. Also, it is to be understood that the invention can assume various alternative variations and stage sequences, except where expressly specified to the contrary. It is also to be understood that the specific devices and processes illustrated in the attached drawings, and described in the following specification, are examples. Hence, specific dimensions and other physical characteristics related to the embodiments disclosed herein are not to be considered as limiting.

A “patient”, “athlete”, or “subject” is a human, and those terms do not imply or require any clinician-patient relationship.

As used herein, the terms “communication” and “communicate” refer to the receipt or transfer of one or more signals, messages, commands, or other type of data. For one unit or component to be in communication with another unit or component means that the one unit or component is able to directly or indirectly receive data from and/or transmit data to the other unit or component. This can refer to a direct or indirect connection that can be wired and/or wireless in nature. Additionally, two units or components can be in communication with each other even though the data transmitted can be modified, processed, routed, and the like, between the first and second unit or component. For example, a first unit can be in communication with a second unit even though the first unit passively receives data, and does not actively transmit data to the second unit. As another example, a first unit can be in communication with a second unit if an intermediary unit processes data from one unit and transmits processed data to the second unit. It will be appreciated that numerous other arrangements are also possible.

As used herein, the term “gait” refers to the manner of locomotion, and in the term of humans, includes the manner of bipedal locomotion, including, for example and without limitation, walking, jogging, running, or sprinting. This includes, in humans, dynamic lower extremity movement pattern in a weight-bearing position with forward movement. The gait cycle includes a stance phase and a swing phase. “Stance” refers to that portion or phase of the gait cycle during which the foot contacts the ground, and can be referred to in terms of a percentage (e.g. stance %) of the gait cycle. “Swing” refers to that portion or phase of the gait cycle where the foot is not in contact with the ground.

As indicated above, when a patient's focus is directed to their extremities as described, this drives their attention internally and increases self-consciousness about body movements and performance which neurologically constrains motor learning. The preponderance of evidence suggests that changing the attention to an external focus where the patient is looking to the effects of their movements on an external feedback cue enhances motor learning.

Real time visual feedback (RTVF) applies and follows this motor learning theory that internalization of a new neuromuscular pattern is enhanced when the patient's focus is directed externally. The patient receives immediate knowledge of their performance, and their attention is directed externally, to the effect their gait pattern changes have on an external cue.

Patients and clinicians, upon early customer discovery, are requesting the ability to extend their training beyond the visit. Clinicians also are asking for dynamic, not static immobile interventions that are more realistic to how a patient really moves and experiences their pain. Clinicians report a desire to know when their patients are having gait trouble out in the world, to better be able to prioritize treatment and be more effective.

Performance at higher levels, including running, benefits from real time visual feedback from sensed information on the user themselves. Training running performance with real-time visual feedback has demonstrated significantly positive results in reducing forces that cause overuse loading injuries, as well as re-educating patterns that could lead to injury and also provide faster responses during rehabilitation. When left unaddressed, it has been reported that 70-90% of those individuals return to their medical providers within 5 years. Real-time visual feedback allows the runner to receive immediate knowledge of their performance and is the fastest integration into the motor learning system versus other forms of bio feedback which do not provide long-lasting results. Video and mirror feedback can be used, however, limitations include the lack of mobility, realism, and provide only a limited quantity of steps the athlete can review.

A wearable system that provides real time feedback for gait functional improvement and running performance addresses the limitations of conventional approaches by providing immediate, specific, and intuitive feedback, which directs the user's focus of attention externally as is recommended according to established learning theory.

According to aspects and embodiments of the invention, provided herein is a gait or running analysis, monitoring, remote training, training in the athlete's environment or patients natural environment or realistic surfaces, functional and performance improvements, reductions in recovery time and rapid detection of prevention strategies, retraining, mobile and portable real time visual feedback gait and optionally monitoring system for a patient, the system comprising: a sensor configured to measure one or more attributes of gait or lower extremity load bearing dynamic function or performance of a patient or athlete; and a controller in communication with a visual, e.g., any wearable (e.g., smart) display, such as a head up display (HUD) output component, the controller configured to, repeatedly (e.g., two or more times), monitor a patient's gait in real-time and to provide real-time feedback to the patient: receive and process information from the sensor representative of one or more attributes of the gait of the patient or athlete; generate a data set corresponding to the information from the sensor representative of one or more attributes of the gait of the patient; compare the generated data set to reference data indicating optimal values for a data set corresponding to the information from the sensor representative of one or more attributes of the gait of a patient or athlete; and cause the display to provide feedback comprising gait analysis based, at least in part, on the comparison between the generated data set and the reference data or the patient/athlete's own baseline data and at least indicating in the feedback if the generated data set is within defined tolerances relative to the reference data.

In other aspects or embodiments, a method of analyzing and/or training for functional and performance improvements in gait, running, and dynamic lower extremity loading in a patient or athlete is provided. The method comprises: placing a sensor configured to measure one or more attributes of gait of a patient on the patient; and, repeatedly, to monitor a patient's gait in real-time and to provide real-time feedback to the patient: receiving and processing information from the sensor representative of one or more attributes of the gait of the patient during one or more physical actions relating to dynamic loading of the lower extremities during activities including gait, walking or running, and optionally, standing performed by the patient; generating a data set corresponding to the information from the sensor representative of one or more attributes of the gait of the patient; comparing the generated data set to reference data indicating optimal values for a data set corresponding to the information from the sensor representative of one or more attributes of the gait of a patient; and causing a wearable display to provide feedback comprising gait analysis based, at least in part, on the comparison between the generated data set and the reference data and at least indicating in the feedback if the generated data set is within defined tolerances relative to the reference data.

In yet another aspect or embodiment, a smart device processor implemented method for analyzing and/or training gait in a patient based on information received from a sensor configured to measure one or more attributes of gait of a patient on the patient adapted to be performed on a portable computing device is provided. The method comprises, repeatedly, to monitor a patient or athlete's dynamic lower extremity movement pattern or gait in real-time and to provide real-time feedback to the patient or athlete: receiving and processing information from the sensor representative of one or more attributes of the gait of the patient or athlete during one or more physical actions relating to dynamic lower extremity movement pattern, gait or walking, and optionally, standing performed by the participant; generating a data set corresponding to the information from the sensor representative of one or more attributes of the gait of the patient; comparing the generated data set to reference data indicating optimal values for a data set corresponding to the information from the sensor representative of one or more attributes of the gait of a patient; and causing a wearable display to provide feedback comprising gait analysis based, at least in part, on the comparison between the generated data set and the reference data and at least indicating in the feedback if the generated inertial, spatiotemportal, force or moment data set is within defined tolerances relative to the reference data.

According to an aspect of the disclosure, feedback devices and systems are provided for training (including retraining) and improving the performance and function of dynamic lower extremity overground movement patterns as well as gait and running movement of a patient. The devices and systems provide a signal, based on sensor data, that serves as an external focus of attention through, e.g., a visual display or through other sensory input able to provide a signal that can serve as an external focus of attention, such as an audio or haptic signal. In aspects, a visual signal may be used as an external focus of attention, to avoid desensitization, which can occur with haptic or causing an excessive cognitive demand such as audio signals. The visual signal can be provided by a head up display (HUD), but for mobile devices and systems the visual signal may be provided by specialized glasses including a visual display, e.g., as described below. In aspects, the sensor device is a force/torque sensor (F/T sensor) and/or a biometric device configured to obtain acceleration, positioning, and/or angular motion data for the subject. Sensor data is provided by a sensor that is worn or otherwise incorporated into a prosthesis or other device placed on the body or in a weight bearing location from the lower extremities of the patient (that is the sensor is a “wearable sensor”), for example, under the foot, on a foot, ankle, knee, leg, hip, or back of a patient. Data is obtained from the sensor, and is converted by a processor or other computing device to a simple signal, e.g., a binary or ternary, string, or integer, signal indicative of the gait of the patient being within or outside of tolerances. For example, the sensor can include commercially available F/T sensors, or motion and movement sensors, such as an inertial measurement unit or in-shoe pressure unit.

Because the device is used for gait training and the improvement of function and performance of dynamic lower extremity over-ground movement patterns, the action and activity includes, for example, walking, jogging, running, or sprinting. In a rehabilitative context, such as after an illness, injury, condition, amputation, or treatment that affects an individual's gait. In aspects, the device and system are mobile, meaning they are configured to be worn on a patient, and can be used in settings outside a clinic. For example, a system may include a force sensor, a computer, and smart glasses. The sensor may be incorporated into a prosthetic or otherwise is worn on or affixed to the patient's body, or under the foot to capture loading patterns, the glasses are worn by the patient, and the computer is carried in a pouch, pack, backpack, or any other suitable carrier on the patient's body or is entirely wireless, and communication is through a smart device for processing. The sensor and glasses are in communication with the computer or handheld processing device, via any suitable interface, wired or wireless, e.g., as described in further detail below.

An F/T sensor is a device that measures components of force and torque (moment) in more than one axis and communicates that data to a computer, e.g., a processor. A common type of F/T sensor is a six-axis sensor that measures all components of force, including force in three axes (Fx, Fy, and Fz) and moment or torque in three axes (Mx, My, and Mz, alternatively Tx, Ty, and Tz). Multi axis F/T sensors are described in the art and are commercially available, such as the i-Pecs Tech Sensor System load cell sensor (commercially available from RTC Electronics of Dexter, Mich. See, Fiedler et al. Criterion and Construct Validity of Prosthesis-Integrated Measurement of Joint Moment Data in Persons with trans-tibial Amputation. J Appl Biomech. 2014 June; 30(3): 431-438) or F/T transducers or sensors from ATI Industrial Automation of Apex, N.C., among many others). F/T sensors can be configured into a prosthesis as described herein, or into a shoe, e.g., as an attachment, or as an insert for a shoe, permitting acquisition and output of force and moment data. Although in many instances a six-axis F/T sensor may be preferred for the detailed data produced, sensors that have lower capabilities (such as Fz only) may be utilized, so long as meaningful data that can be used to determine the presence and/or quality of steps and other relevant activities can be derived from the output data from the sensor. Data can be transmitted from the F/T sensor by wire (e.g., USB, Ethernet, etc.), or wirelessly (e.g., by Bluetooth).

An inertial measurement unit is an electronic device that measures and reports movement and positioning data (e.g., an inertial data set), velocity, acceleration, and angular rate using a combination of accelerometers and gyroscopes. Inertial measurement units are commonly used in inertial navigation systems for aircrafts. When used in an aircraft, an inertial measurement unit is used for detecting a current rate of acceleration in multiple axis (e.g., acceleration in the x, y, and z directions) with one or more linear accelerometers, and for detecting rotational attributes like pitch, roll, and yaw, using one or more or angular accelerometers and/or gyroscopes. Measurements from the accelerometers and gyroscopes can also be used for calculating changes in position of the device. In one example, a commonly used inertial measurement unit design includes three accelerometers positioned to measure acceleration along three axes which are orthogonal to one another (e.g., the x, y, and z axes). The inertial measurement unit also includes three gyroscopes placed in a similar orthogonal pattern for measuring rotational position of the sensor device around each of the axes. Information from the six sensors can be combined together using different positioning algorithms to determine an absolute or relative position of the wearable sensor device.

While acceleration and angular momentum information can also be collected from multipurpose electronic devices, such as smart phones, which use the innate mobile device accelerometer technology to obtain angular motion data, such smartphone devices are not meant to be worn on the person's torso and therefore require assistance of an additional person to collect movement data, thus requiring at least a specialized harness. In addition, data received from accelerometers on smart phone devices generally do not have the requisite level of accuracy provided by the wearable sensor devices disclosed herein. For example, the innate accelerometer technologies used by smartphone devices may not have an appropriate range or specificity to measure data for some assessments. As will be appreciated by one of ordinary skill in the art, accuracy and precision of the assessments are needed to identify proper gait and dynamic lower extremity over-ground movement patterns from measured movement data.

The sensor device desirably is sufficiently lightweight and of small enough size to not interfere with a patient's range of motion. The sensor device allows for real-time data acquisition and analysis of the subject to produce real-time output to a patient without needing input and/or analysis of collected data by a clinician. Another advantage is that assessments based on information measured by the sensor device can be performed at any location, meaning that individuals do not need to travel to a specialized lab or clinic to perform training activities. In addition, they do not rely on subjective visual analysis of movement patterns.

In aspects, the sensor device communicates, e.g., wirelessly, with a nearby computing or communication device (referred to herein as an intermediary device), such as a portable computing device, cellular telephone, smart phone, or Internet gateway device, to drive data collection, analysis and output to a wearable display, such as a smart display or an HUD, or any other wearable visual smart display technology. Visual cues directed to training are superior to audible and haptic feedback as they are the quickest to be sensed by patients, and are not subject to desensitization and interference. This intervention correctly applies the true desired external focus of attention that provides a direct “effect” of the incorrect movements and correct patterns on an external display versus simply providing raw data. This improves the individual's ability to integrate the training into their own automatic error detection processes. In aspects, the intermediary device comprises a controller configured to implement software for receiving and processing information from the sensor device and for providing feedback that indicates to the user the effect of their movements, e.g., via a wearable visual display, e.g., an HUD, or other wearable visual smart technology, based on the movement information. In aspects, feedback includes displaying indicia on the display indicative of the quality of the patient's gait, which is a real time use of the motor learning theory external focus of attention and provides the “effect” of the patient's movements qualitatively on an external cue versus displaying raw sensor data. In one aspect, the indicia indicates proper gait within predetermined or on the individual's own baseline tolerances and indicates improper gait dynamic lower extremity over-ground movement patterns falling outside those tolerances, for example, with a red signal indicating improper gait and a green signal indicating proper gait, for example, displayed on a display in the peripheral vision of the patient in a completely portable fashion untethered or requiring the patient to be indoors. In other aspects, the indicia indicates proper gait within predetermined tolerances, and indicates improper gait, specific deficiencies, and/or specific corrective actions to be taken to correct the improper gait. For example, providing a green signal indicating proper gait, and one or more additional signals, such as solid red signal if a parameter is below ideal, and a solid yellow signal if a parameter is above ideal. That said, in training, in aspects, it may be preferable to provide the simplest feedback.

In one aspect, the signal is binary or ternary, but the system provides a graded transition between signals as the patient's activity approaches optimal. For example, in a binary system, the out of tolerance signal may be yellow, and the in-tolerance signal may be green, with a stepwise transition from yellow to green, for example, in two, four, eight, 16, 32, 64, or 128 graded steps, as the patient's walking or running motion approaches an appropriate gait.

In one aspect, the signal to the patient provides an external focus of attention. Audible corrective measures or feedback typically drives a patient's focus to internally regard their limb and corrective measures, and specifically how a limb is moving. Haptic signals often are missed. Therefore, in one aspect, the signal to the patient is visual, and in aspects, the signal provided to the patient via the display is binary (that is, providing only two signals, such as two different color signals), indicating the effect of their corrections and directing them to that effect on the colors on the wearable screen within tolerances or outside of tolerances. In other aspects, the signal is ternary, e.g., sending three different colors to the display, one color indicating “above tolerances”, a second color indicating “below tolerances”, and a third color indicating “within tolerances”, such as the above-mentioned red, yellow, and green scheme.

The described colors are merely illustrative of the multitude of possible indicia available to those of ordinary skill, including: colors, flash patterns, shapes, positioning on the display, text, or any other shape, icon, pattern, sounds (e.g., in a built in speaker, ear-bud or any sound transducer), vibrations, etc., that can be indicative of any measurable feature relating to gait. The focus is directed to the effect of movement on an external feedback cue, versus isolating limb actions which is internally directed focus.

According to one aspect of the disclosure, baseline data is obtained from one or more sources, including parameters obtained from literature, parameters from one or more individuals other than a patient, and/or from the patient walking in a correct manner, e.g., as determined by a clinician. The baseline also may be detected via processing at that individuals baseline for the desired dynamic lower extremity over-ground movement pattern that needs training. Data is then obtained from the sensor, such as from an F/T sensor in a prosthetic or in a wearable device, such as a shoe, e.g., in the sole of a shoe, for example, as a shoe insole, shoe outsole, cover, or other attachment, or from an inertial measurement unit worn by the patient or incorporated into a prosthetic device.

The sensor device and feedback system is worn either in a clinic, or in the real world, and provides direct, real-time feedback to the patient, thereby training the patient to walk, jog, run, or sprint with a proper dynamic lower extremity over-ground movement patterns. Measured results can be stored in a database of inertial data sets (e.g., .csv or similar files) either automatically or manually at the request of a clinician, programmer, or system administrator. In some instances, measurement is performed on the subject two or more times, such as during an initial patient evaluation and after the training has been followed for a few days or weeks. Baseline data, or a data set acquired at the earlier or initial time point can be compared to a data set acquired at the later time. The deviation between the data sets at different time points can be analyzed either automatically by a computing device (e.g., the intermediary device), or by a user, technician or administrator, to decide whether actions or activities (e.g., a recommended treatment regimen) or tolerances of the device (e.g., the criteria used to distinguish gait falling within specifications or outside of specifications) need to be changed to achieve a desired outcome, such as improvement in gait. Results of the analysis are used to improve future treatment recommendations either for a particular subject or for all subjects using the devices and systems described herein.

In the examples provided herein, the quality of a patient's gait is measured by or represented by the stance % criteria. That is, raw sensor data, e.g., Fz data, is converted by one or more computer processes to produce a stance % value. An optimal stance % range of values for a desired gait is determined, and during a therapeutic session, when a patient walks or runs, a signal is sent to the patient when the patient's gait is within the optimal range, and a different signal is sent when the patient's gait is outside the optimal range. Stance % is one of many criteria that may be used as a measure of the quality of the patient's gait. Raw data from the sensors, e.g., spatial-temporal force or moment data, can be used to generate values including, without limitation: stride (heel to heel), stride length, cadence, force (e.g., Fz), torque of knee, and any other useful measure of gait quality. Criterion, such as cadence and stance %, can be combined. Two or more sensors can be placed on a patient's body and/or incorporated into a device, to generate useful data.

### Sensor Device

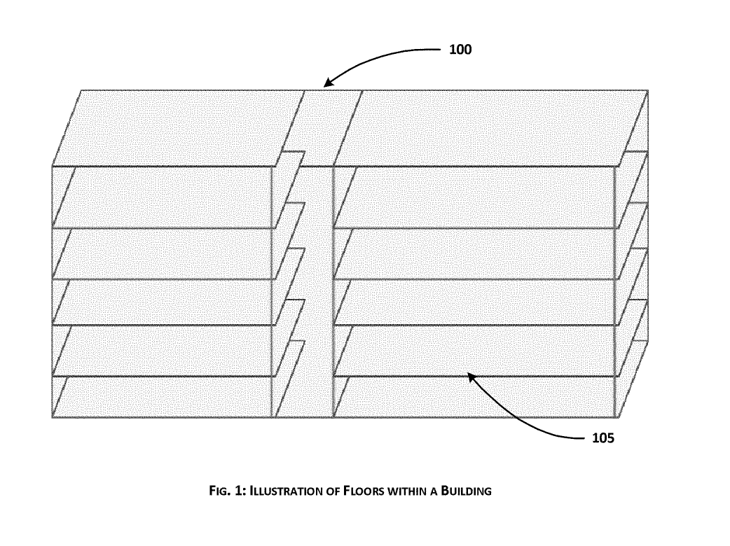

FIGS. 1A-1C show an exemplary sensor device 10 for collecting movement information for a subject. In some examples, the device includes a housing 12 enclosing circuitry for collecting force, torque, and/or movement information. The housing 12 can be formed from a lightweight, rigid material such as plastic or brushed aluminum. In some instances, the housing 12 can include various removable covers or other openings for accessing interior components of the device 10, such as batteries, sensors, memory cards, and other items. The housing 12 can also include one or more ports 14, such as a USB, Ethernet, Thunderbolt, or Firewire port, for wired connection between the sensor device 10 and other computing devices. In some instances, the device 10 can include one or more visual indicators 16, such as LEDs, on or extending through the housing 12 for conveying information to a user. For example, a green-color LED 18 can be turned on when the device is ready to use. A red or yellow colored LED 20 can be turned on to indicate to the user that the device 10 is not ready to collect data if, for example, the device battery is depleted or if the device 10 does not include sufficient memory to record assessment measurements. In some instances, a visual indicator 16 can also be used to indicate when the device 10 is in wireless communication with another computer device and/or when the device 10 is uploading data to another computer device.

In some examples, the sensor device 10 includes a harness, band, adhesive patch, or another connection mechanism for affixing or mounting the sensor device 10 to the patient. In other examples, the sensor device 10 may be incorporated into a prosthetic or wearable device, such as a shoe. In one aspect, device 10 includes a strap for attaching the device 10 to the user's waist, torso, or hips. The strap includes a connector, such as a buckle or hook and loop fastener (e.g., Velcro™), for attaching ends of the strap together to hold it in place, e.g., around the subject's waist. In other examples, the sensor device 10 can be attached to a necklace or collar and worn around the subject's neck. In still other examples, the device 10 can be affixed to the individual's clothing using a clip, clasp, or similar fastener.

With specific reference to FIG. 1B, internal circuitry of a sensor device 10 is illustrated. The sensor device 10 includes electronic circuitry, such as an F/T sensor 30, enclosed within the housing 12 for measuring movement information of the subject wearing the device 10. The device 10 also includes a storage module 36, comprising transitory or non-transitory computer readable memory for storing information collected by the F/T sensor 30.

The F/T sensor 30 comprises movement sensors, such as a three-axis force sensor 32 and a three-axis torque sensor 34 of a six-axis F/T sensor. In some examples, the F/T sensor 30 includes three orthogonally positioned accelerometers and gyroscopes. With specific reference to FIG. 1C, internal circuitry of a sensor device 10 is illustrated. The sensor device 10 includes electronic circuitry, such as an F/T sensor 30, enclosed within the housing 12 for measuring movement information of the subject wearing the device 10. The device 10 also includes a storage module 36, comprising transitory or non-transitory computer readable memory for storing information collected by the F/T sensor 30.

With specific reference to FIG. 1C, internal circuitry of a sensor device 10 is illustrated. The sensor device 10 includes electronic circuitry, such as an inertial measurement unit 40, enclosed within the housing 12 for measuring movement information of the subject wearing the device 10. The device 10 also includes a storage module 36, comprising transitory or non-transitory computer readable memory for storing information collected by the inertial measurement unit 40.

The inertial measurement unit (IMU) 40 comprises movement sensors, such as one or more single axis or multi-axis accelerometer(s) 42 and one or more gyroscope(s) 44. In some examples, the inertial measurement unit 40 includes three orthogonally positioned accelerometers and gyroscopes. An accelerometer measures acceleration. Most accelerometers can also measure tilt. The accelerometer was originally a large device, but with the continued advances in the field of micro-electromechanical systems (MEMS) technology, accelerometers are presently available in sizes of less than 1 or 2 mm, with 3-axis measurements. A gyroscope measures orientation. In one aspect of the device 10, a gyroscope is used to determine changes in the orientation of the subjects' body to help identify the physical activity being performed. Gyroscopes based on MEMS technology are now also widely commercially available. Commercial chips that combine a 3-axis accelerometer and a 3-axis gyroscope are commercially available. One non-limiting example of a useful device is the WAX9 IMU, commercially available from Axivity Ltd. of York, UK, having accelerometer and gyro functionality as well as Bluetooth connectivity, a magnetometer, a barometric sensor, a temperature sensor, a micro-USB connector, suitable firmware, and a processor.

In some examples, the sensor device 10 also includes a timer or clock. The timer is used to record a time when certain data is collected. The acquisition time can be stored by the storage module 36 along with the collected data for providing a time-stamped record of physical activities performed by the subject.

As shown in FIGS. 1B and 1C, the sensor device 10 also includes a communications module 38 for wired or wireless communication with an external computing device. In some examples, the communications module 38 transmits collected data from the device 10 to another computer device automatically substantially in real time. In other examples, sensed information is collected and stored in the storage module 36 and uploaded to the computer device as a batch file transfer. Uploads can occur periodically according to a predetermined schedule or, for example, in response to an event, such as a request from the external computer device or when the sensor device 10 is in proximity (e.g., within range for file transfer via short-range wireless data transmission) to the computer device. In some examples, the communications module 38 is a wireless transceiver, such as a transceiver employing IEEE 802 wireless networking standards, by Bluetooth®, Wi-Fi, ZigBee, LAN, WAN, or cellular connection, or combinations thereof. Wired data transmission may occur via USB, Firewire, or Ethernet networking standards.

In aspects, the wearable sensor device also includes power management components, such as a rechargeable battery 50 and associated control circuitry. For example, the control circuitry can monitor battery parameters such as charge remaining. In some examples, the device 10 provides output to a user when the battery 50 needs to be recharged or when the battery 50 is too depleted to continue data collection.

### Prosthesis

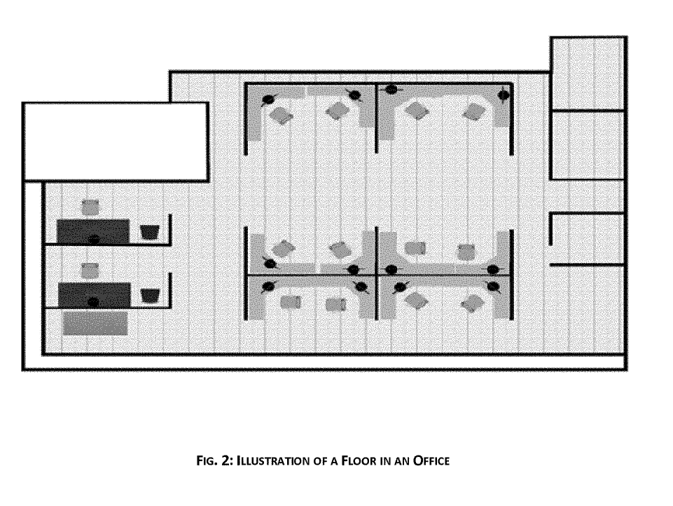

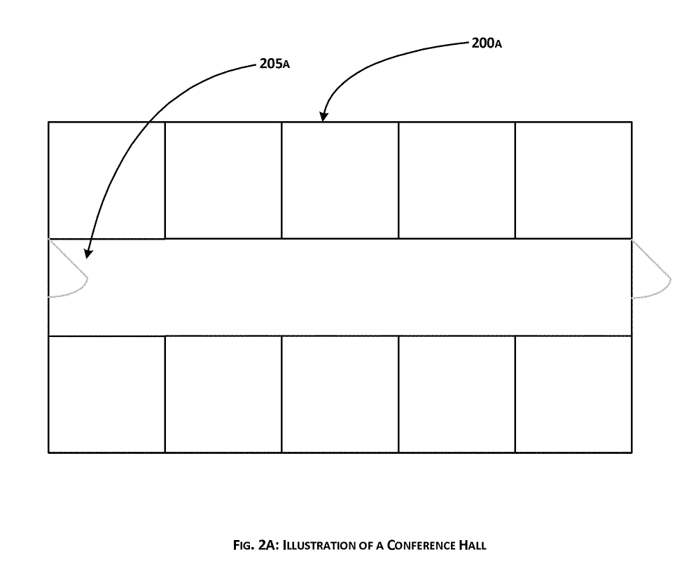

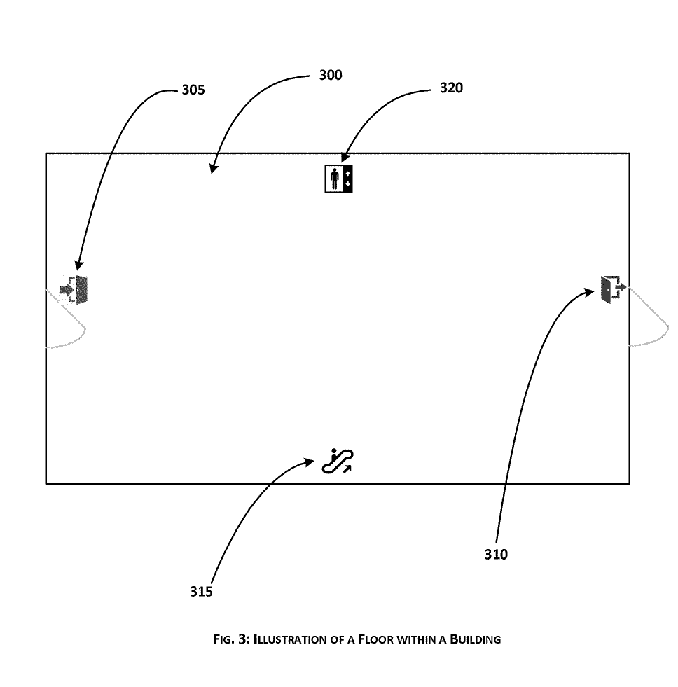

With reference to FIG. 2, in aspects, a trans-tibial prosthesis device 51 is depicted in schematic and not in scale for ease of depiction. The device 51 comprises: a socket 52, configured to receive a stump of a patient, an interface 53, a pylon 54, and a foot portion 56. A sensor 58, such as an F/T sensor, as described herein, is provided in-line with the pylon 54. Sensor 58, such as an i-Pecs™ sensor, may comprise a wired or wireless communications interface as described herein.

### Movement Analysis System

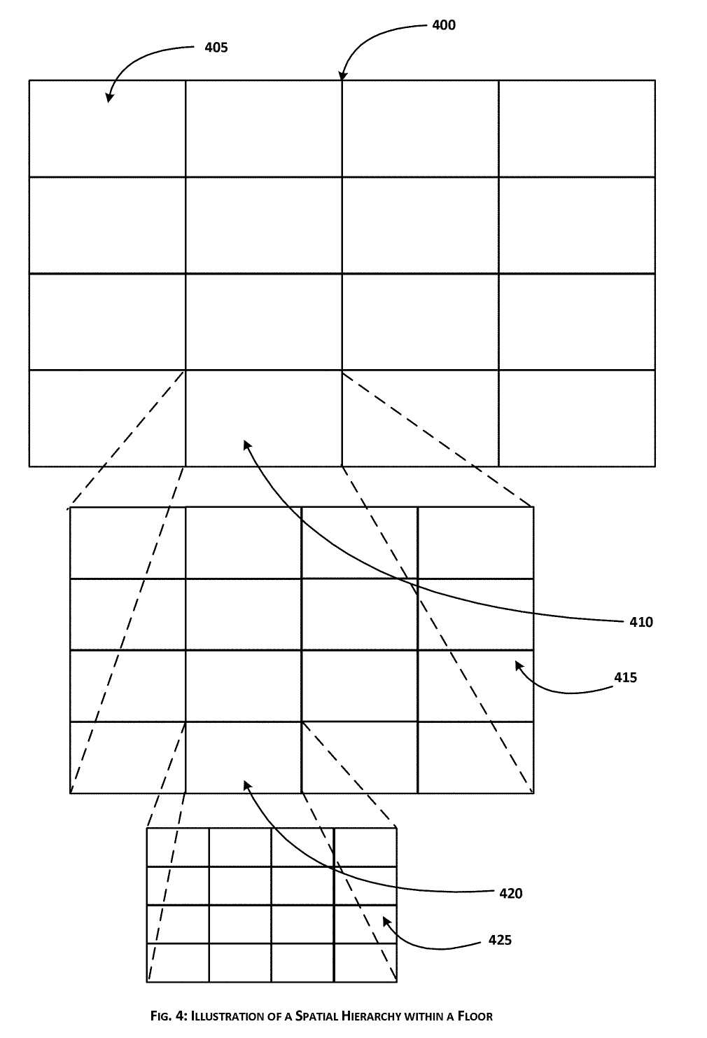

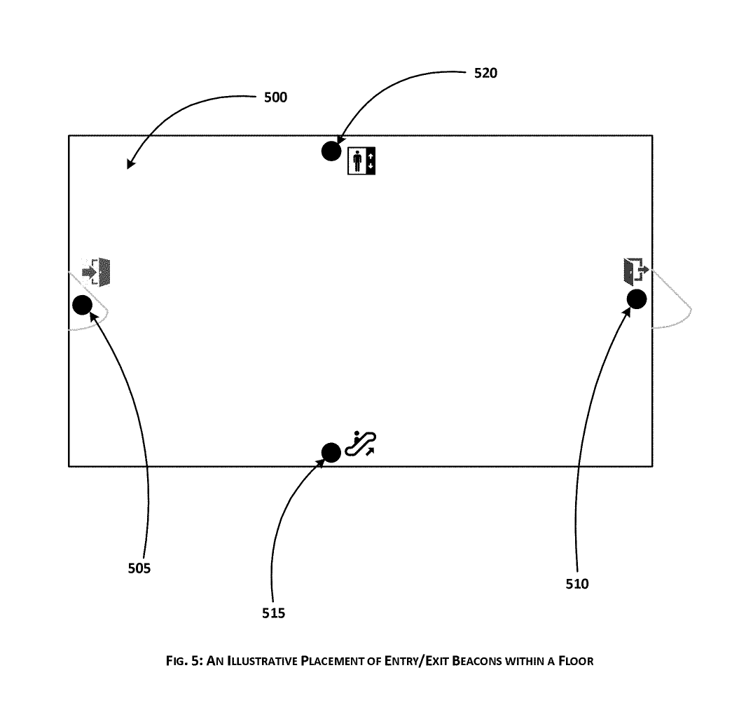

A movement analysis system 100 including one or more sensor devices 10 is shown in FIG. 3. The movement analysis system 100 can be configured to obtain data from the sensor device(s) 10 for the purpose of sensing, analyzing, and training the gait of a patient. Data for the patient can be transmitted either directly or indirectly to a central device or server. For example, as shown in FIG. 3, the data is received by an external computer network 110 comprising one or more computing devices (computers) 112 in communication with storage devices 114 comprising computer readable memory comprising databases of movement results for various subjects.

Intermediary Device

In some examples, data from the sensor device(s) 10 is first transmitted to an intermediary device 116, which receives data from the one or more sensor devices 10 and transmits the data to the computer network 110. The intermediary device 116 can be a dedicated electronic device comprising non-transitory computer readable memory with instructions for receiving, processing, communicating/transmitting, and providing feedback about the information from the one or more sensor devices 10.

In other examples, the intermediary device 116 is a multipurpose electronic or computer device capable of performing processes for data collection and analysis (referred to herein as a computer). In the context of computing, a process is, broadly speaking, any computer-implemented activity that generates an outcome, such as implementation of a mathematical or logical formula, operation, or algorithm. For example, the intermediary device 116 can be a portable computer device, laptop computer, tablet, microcomputer (e.g., Raspberry Pi or Arduino), or smartphone (such as an Apple iPhone or a Samsung Galaxy). Other examples include a workstation, a server, a laptop, a tablet, a smart device, a web-enabled telephone, a web-enabled personal digital assistant (PDA), a microprocessor, an integrated circuit, an application-specific integrated circuit, a microprocessor, a microcontroller, a network server, a Java™ virtual machine, a logic array, a programmable logic array, a micro-computer, a mini-computer, a large frame computer, or any other component, machine, tool, equipment, or some combination thereof capable of responding to and executing instructions. The portable computer device can be configured to execute instructions from a software application (e.g., an App) which controls health monitoring and collection of data from the sensor device(s) 10. For example, an App can be one or more of an operating system (e.g., a Windows™ based operating system), browser application, client application, server application, proxy application, on-line service provider application, and/or private network application. The App can be implemented by utilizing any suitable computer language (e.g., C\C++, MATLAB, UNIX SHELL SCRIPT, PERL, JAVA™, JAVASCRIPT, HTML/DHTML/XML, FLASH, WINDOWS NT, UNIX/LINUX, APACHE, RDBMS, including ORACLE, INFORMIX, and MySQL). The App can comprise health, fitness, and/or physical movement analysis software. In some instances, the App can be downloaded to the device 116 from an external source, such as the external computer network 110. Following initial installation of the App, the device 116 can be configured to receive instructions, updates, or additional software from the external source either according to instructions included with the App or in response to a request from the external source.

In other examples, the intermediary device 116 is another medical, exercise, or patient monitoring device located in close proximity to the subject. For example, various types of exercise and medical equipment may include microprocessors for controlling device function. Instructions for receiving, processing, and providing feedback about sensed movement information from the one or more sensor device(s) 10 can be loaded or downloaded to any such devices for implementing the patient monitoring and feedback systems discussed herein.

Controller

In some examples, the intermediary device 116 comprises a controller 118 for executing functions related to receipt, analysis, and transmission of sensed movement data. In some examples, a controller is a central processing engine including a baseline processor, memory, and communications capabilities. For example, the controller 118 can be any suitable processor comprising computer readable memory 120 and configured to execute instructions either stored on the memory 120 or received from other sources. Computer readable memory 120 can be, for example, a disk drive, a solid-state drive, an optical drive, a tape drive, flash memory (e.g., a non-volatile computer storage chip), cartridge drive, and control elements for loading new software.

In some examples, the controller 118 includes a program, code, a set of instructions, or some combination thereof, executable by the device 116 for independently or collectively instructing the device 116 to interact and operate as programmed, referred to herein as “programming instructions”. In some examples, the controller 118 is configured to issue instructions to one or more of the sensor devices 10 to initiate data collection and to select types of measurement information that should be recorded. In other instances, the sensor device(s) 10 is configured to automatically transmit all sensed movement data to the intermediary device 116 either in real time or at periodic intervals without first receiving initiation instructions from the controller 118 to initiate sensing and data transmission.

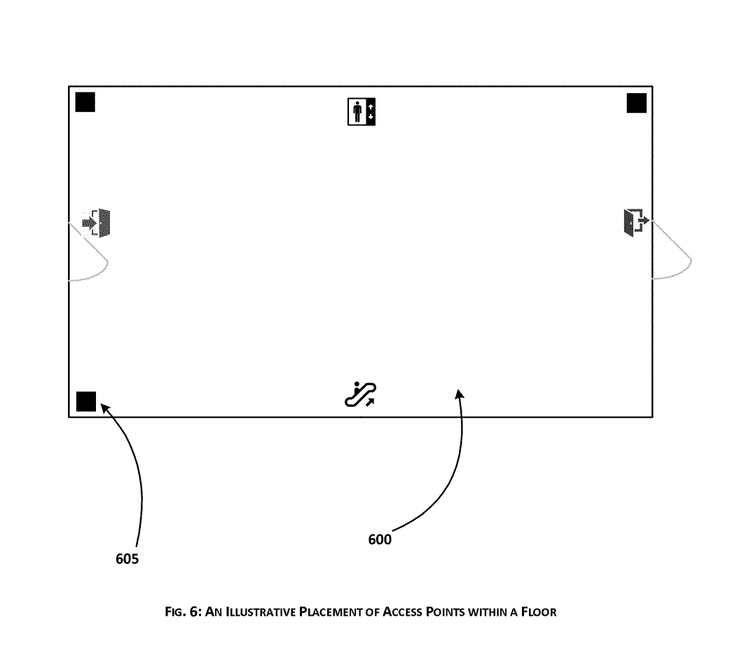

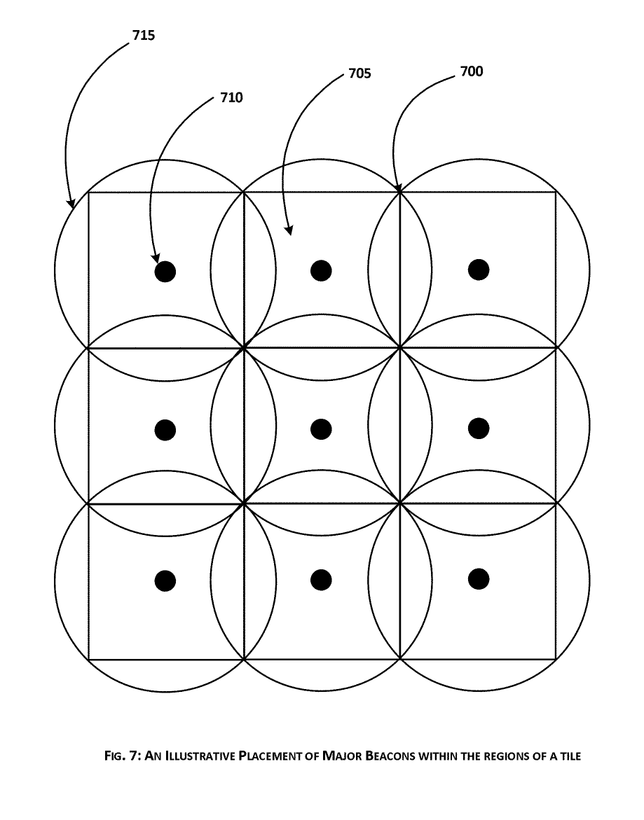

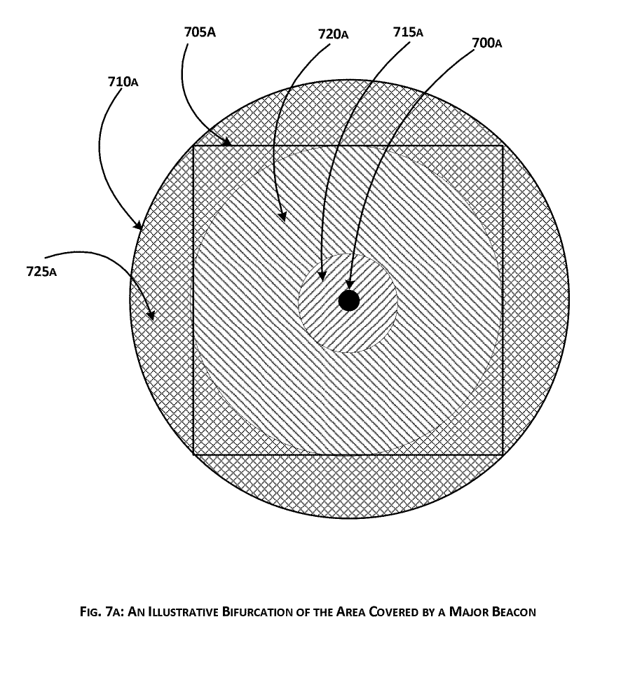

In either case, as will be discussed herein, the controller 118 is configured to receive and process movement information from the sensor device(s) 10 for activities performed by the subject. Processing can include applying filters and other techniques for removing signal artifacts, noise, baseline waveforms, or other items from captured signals to improve readability. As discussed in greater detail in connection with the discussion of FIGS. 4 and 5, processing information includes data analysis techniques, such as quantifying various movement parameters based on received data, corroborating or calibrating data from multiple sources, and/or analyzing generated movement parameters to draw conclusions about the subject.

In one example for analyzing received data, the controller 118 is configured to compare one or more inertial data sets obtained from the sensor device(s) 10 with reference data comprising one or more reference inertial data sets stored on the computer readable memory or received from external sources, such as the computer network 110. For example, the reference inertial data sets can be stored on the storage device 114 and transmitted to the intermediary device 116 via the computer network 110. In some examples, the reference inertial data sets include average parameter values or target parameter values for individuals having similar physical characteristics to the subject. The controller 118 can be configured to determine one or more deviations, if any, between the inertial data set(s) and the reference inertial data set(s), and, if one or more deviations is present, generate a list of one or more activities or actions (e.g., a recommended treatment regimen) that the subject could perform as a corrective measures to address the identified deviations between the subject's inertial data set and the average or target data set for similarly situated individuals. Possible corrective actions, in the form of a treatment regimen or treatment protocol, can also be stored on a database on the storage device 114 and transmitted to the intermediary device 116 by the computing network 110 when required.

Communications Module

In some examples, the intermediary device 116 comprises a communications module 122 associated with the controller 118. In that case, the controller 118 is configured to cause the communications module 122 to transmit the raw, processed, or analyzed data from the wearable sensor device(s) to remote sources, such as the external computer network 110. In other examples, the data is uploaded from the intermediary device 116 to an internet webpage or other remotely accessible database.

The communications module 122 comprises a short range data transceiver for communication with the communications module 38 (shown in FIG. 3) of the sensor device(s) 10. For example, the short range data transceiver may be a Bluetooth® transceiver, Zigbee transceiver, or similar data transmission device, as are known in the art. In other examples, the short range data transceiver can be a radio-frequency (RF) near-field communication device. In other examples, the communications module 122 comprises a wired data transmission interface. In that case, the sensor device 10 can be connected to the intermediary device 116 using a USB cable or similar data transmission cable. The communications module 122 can also include a long-range wireless data transceiver 124 for communication with the computers 112 of the external computer network 110. For example, long range data transmission can use WiFi, cellular, radio frequency, satellite, and other known data transfer devices and protocols. Communication between the sensor device(s) 10, the external computer network 110, and, if present, the intermediary device 116 can be encrypted by any useful method. In that case, the communications module 122 can be configured to receive encrypted data from the sensor device 10 and process the encrypted data to remove encryption so that the received device can be analyzed. The communications module 122 can also be configured to encrypt data prior to long-range data transmission to the external computer network 110. For example, the devices 10, 112, 116 can use encryption, data redaction, and/or security mechanisms to ensure data privacy and that the system comports with privacy standards, such as the U.S. Health Insurance Portability and Accountability Act (HIPAA) standards.

Input/Output Components

In some examples, the intermediary device 116 further comprises an input component 126 and an output component 128 in communication with the controller 118, which allow the user to interact with and receive feedback from the intermediary device 116. The input component 126 includes one or more of a keyword, touchpad, computer mouse, trackball, or other data entry accessory, as are known in the art. In other examples, input components 126 include a microphone for capturing audio data entry by a user or optical or motion sensors for capturing gestures performed by the user. The input component 126 can be used to enter information about the subject which can be used to analyze the measurement data and/or to assist in gait analysis and training regimens. For example, information about the subject's gender, age, height, weight, activity level (e.g., recreationally active, occupationally active, soldier, elite athlete), and other relevant information can be entered via the input component 126. The input components 126 can also be used to interact with a user interface by, for example, being able to toggle through instruction screens for positioning the sensor device 10 on the subject and for performing different types of activity assessments. User interface screens that can be shown on a visual display and used for entering information and guiding a user in collecting information about a subject are shown in FIGS. 6A to 6E and discussed herein.

In some examples, the input/output components 126, 128 is a touch screen display. In other examples, output component 128 includes a visual display, speakers, haptic output devices, and/or other types of feedback devices as are known in the art. The output component 128 can provide feedback to the clinician or subject about the subject's physical condition and, in particular, feedback based on movement information captured by the sensor device 10 to guide the patient to reproduce a proper gait.

In one aspect, the output components 128 include a “head up display” (HUD), as are broadly-known in the art, or any wearable visual display or smart display technology. Commercial examples of such displays include: Google Glass 2.0, Recon Jet™ (Recon Instruments), Glass (X, Mountain View, Calif.), and Vuzix M100 or M300 (Vizix Corporation, West Henrietta, N.Y.). Wearable displays can be any form, so long as they place a visible display, e.g., on glasses or another wearable device, capable of producing an indicia of the status of a patient's gait. Depending on the nature of the indicia desired and/or necessary to guide the patient, the display may be as simple as providing small LEDs or similar structures, or fiber optic displays, or as complex as a color display capable of displaying complex images. Optionally, haptic (e.g., vibration) sensations or audible signals also may be produced by the display device, or a connected transducer, such as an earbud or vibrator. Certain display devices also are capable of producing input data relating to movement and position of the patient, including accelerometer and gyroscope functionality, which can be used in addition to a dedicated sensor device worn on the waist, hips or torso—so long as a relevant measurement of one or more aspects of the gait of the patient can be ascertained from such input data.

In addition to providing feedback, in some examples, the controller 118 is configured to cause the output component 128 to provide visual or audio instructions to the user or subject related to the movement assessment being performed, such reminders that assist the patient to remember what to focus on when walking. More than one output component 128 may be utilized, such as a screen for a clinician in addition to the display device.

The components of the sensor device 10, intermediary device 116, and external computer devices 112 can be combined in various manners with various analog and digital circuitry, including controllers, filters, ADCs (analog-digital chips), memory, communication devices and/or adaptors. Especially, but not exclusively with respect to the sensor device 10, as devices become smaller and processors become more powerful and use less energy, it is possible to integrate many more sensors, such as microelectromechanical systems (MEMS) or nanoelectromechanical systems (NEMS), onto single chips. MEMS accelerometers, gyroscopes, gas sensors, thermometers, humidity sensors, and magnetometers are readily available from commercial sources and/or are abundantly described in the art. Technologies such as package on package (PoP) and system on a chip (SoC) integrated circuit packages allow manufacture of very small devices with significant capacities. For example, smart phones use PoP technologies to stack memory and processors in a very small volume. One example of a SoC is a microcontroller (MCU), which is a small computer on a single integrated circuit typically containing a processor core, memory, and programmable input/output peripherals. MCUs also may include timer module(s) and analog-to-digital converter(s) for, e.g., converting analog sensor output to a digital signal.

External Database

With continued reference to FIG. 3, in some examples, the intermediary device 116 is in communication with the storage device 114 of the external computer network 110. For example, the intermediary device can receive information including patient information and reference data sets from databases stored on the storage device 114. For example, the storage device 114 can comprise a database of patient electronic health records for subjects. A health record contains personal information for the subject such as a subject's name, age, weight, height, body mass index (BMI), and blood pressure. A health record can also contain information about assessments previously performed by the subject or about a subject's history of past injuries. The intermediary device 116 can, for example and without limitation, store, communicate the personal information, and combine the personal information with the inertial data set information for communication to other external computer devices, such as the computer device 112. In some examples, the intermediary device 116 is also configured to redact private information from the personal information prior to communication of the personal information. The received patient information is used by the computer device 112 to improve analysis of the sensed movement information.

The storage device 114 may comprise a database of reference data sets with movement information for a wide range of subjects, or parameters based on other sources, such as from research studies. The database can be used to obtain reference datasets for other individuals with similar characteristics (e.g., physical characteristics, occupational or activity level, and/or medical history) as the subject. Physical measurements for the subject can be compared with reference data sets for improve specificity and accuracy in gait analysis and feedback. In some instances, reference data sets are based on statistical (e.g., average) values for segments of a population (e.g., segments of the overall population with physical characteristics similar to the subject) or for the population generally. In other examples, a set of reference data sets is provided from the database for individuals with varying degrees of proper or improper gait. In one example, the database includes a reference data set or individual classified sets for an individual with fully- and properly-functional legs (no injury history), as well as for individuals with one or two prosthetic lower extremities, different types of prosthetics (above the knee, below the knee, below the ankle, etc.), and/or classified by one or more motions characteristic of a proper or improper gait. The measured inertial data set for the patient can be compared to inertial data sets for others, or other reference values, to assess progress or to assess the type of corrections needed to achieve a desired target motion. As information for different subjects is obtained, processed, and analyzed, the database can be expanded in an iterative manner to improve specificity and accuracy in gait training.

### Information Collection Processes

FIGS. 4, 5, and 7 are flow charts illustrating processes for monitoring movement of the subject using the sensor device, processing and analyzing information sensed by the sensor device, and providing feedback to a patient via the display relating to gait, as described above. These processes are performed using the wearable sensor device(s), intermediary device, and external computer network, e.g., as shown in FIG. 3. The processing and data analysis techniques discussed herein can be performed by the intermediary device and/or by remote computer devices of the external computer network. In some instances, processing and data analysis functions are distributed between multiple computer processors on different devices. In one example, initial processing and data analysis, is performed by the controller of the intermediary device and feedback is sent directly to the display. More sophisticated data analysis and reporting functions can be performed on remote computer devices of the external computer network (e.g., in the cloud).

As shown in FIG. 4, the sensor device or intermediary device guides the user or subject through an initial setup process. In the example shown by box 210, during the setup process, the user is instructed to input patient demographic information about the subject's physical condition and other information. For example, in response to requests by the wearable sensor device or the intermediary device, the user or subject provides demographic information (e.g., age, weight, height, dominant limb) and/or activity level/type information for the subject. The subject can also be identified as a member of a particular group of interest. For example, the subject may identify that he or she is a member of a particular sports team, military branch, cohort, etc. Assessment results for identified members of a group can be compared together during data analysis.

Based on the entered information, as shown at box 212, the sensor and/or intermediary device is configured to select a battery of evidence-based assessments to collect movement data for the subject. For example, one or more force or torque value (e.g., Fx, Fy, Fz, Mx, My, and/or Mz), is obtained from the sensor and/or a parameter is calculated (e.g., percent of time foot is contacting the ground (% stance), AUC of any value, peak forces or moments, range of any force or moment, proximal (e.g., knee) forces, such as pMy or pMz, or distal (e.g., ankle) forces, such as dMx) from the raw data. As illustrated in the flowchart of FIG. 5, based on input from the sensor, a step is identified from one or more parameters, and is distinguished from motions (or lack thereof) that does not indicate stepping. For example, periodic fluctuations in Fz might indicate a step motion (see, exemplary data in FIG. 6).

As with distinguishing stepping motions from non-stepping motions, any combination of measured or calculated forces during stepping can be compared to reference values, and if such measured or calculated forces fall within predetermined tolerances based on the reference values, a signal is passed to the HUD indicating the patient's walking motion is within tolerances. If such measured or calculated forces during stepping fall outside predetermined tolerances, a signal is passed to the HUD indicating the patient's walking motion is outside tolerances, indicating that the patient should self-correct. As training advances, and a patient spends increased time within tolerances, a clinician or a software process can decrease the range of tolerable motions so that the patient can further fine-tune her gait.

A process for analyzing the quantified or normalized values to provide feedback to the user or subject for injury prediction and treatment recommendation is shown in FIG. 5. As shown in FIG. 7, the obtained parameter value(s) may be analyzed by comparing the derived values to measurements obtained from other subjects, the patient, or other sources, such as from published data or parameters, and stored in a database. In some examples, the other subject data includes average values based on measurements for a group of similarly situated individuals. In other examples, reference values are calculated or derived from data for the general population.

Referring to FIG. 7, as shown at box 310, the process begins when the values for the measurements of interest are received. As described herein, the values are derived from movement data collected by the sensor device(s) worn by the subject while performing the assessment(s). In one example, the received values include values derived from multiple data sets, such as a first data set and a second data set. The process also includes identifying characteristics of the subject so that reference values may be obtained, as shown at box 312. Subject characteristics include the subject's age, history of previous injuries, and/or the subject's activity level and/or level of occupational activity. In one example, this characteristic information is provided by the user or subject as discussed above. In other examples, subject information is automatically downloaded from a remote source, such as a patient health record.

As shown at box 314, benchmark or reference data sets are obtained from external sources, stored on one or more databases of the computer network, and downloaded to the computer device or intermediary device as needed. In one example, derived force, torque or stance measures are compared to benchmarks determined through analysis of collected normative data. Specific entries or values from the database or data set are selected based on the provided demographic information about the subject.

As shown at box 316, the measured reference values for the subject are then compared to the reference values from the database. The results of the comparison can be used to determine a derivation between the measured data and reference data sets. Comparison of one data set to another can be accomplished by any method, for example and without limitation, by differencing methods. A variety of other methods and data formats are amenable to such comparisons. For example, a computer process, such as a table, a matrix, a statistical representation, an object, an equation, an image, compressed data, and combinations thereof can be used in manners which are apparent to those of ordinary skill in the art.

As shown at box 318, the derived data set and/or results of the comparison between the data set and reference values are used for gait analysis and training, and thus output is transmitted to an HUD so that the patient can self-correct if outside of tolerances during walking or other activity relating to gait. For example, reference values can be viewed as cutoff points for tolerances associated with a certain training activity.

As shown in box 320, the training process is repeated as necessary, and as shown in box 322, a report can be generated.

### EXAMPLE 1

It is the purpose of this example to develop and examine the effects of a real time mobile visual feedback (RTMVF) gait training system for individuals with amputation. A secondary purpose is to examine the effects of this program on improving gait performance (symmetry and frontal plane pelvic motion) pain and functional measures. In addition, findings of this project will expand the knowledge of how well this form of training can affect the retention. Removal of feedback in a fade out pattern to improve internalization, as well as a systematic training protocol and an external focus of attention (the effects of movement seen on the feedback display) can be used. These have both been demonstrated as training strategies to improve motor learning, however have not been widely used in the amputee population for gait retraining. One of the primary criticisms of the previous studies is limitation due to the expense of instrumented treadmills and motion capture systems to assess gait kinematic and temporal-spatial outcomes of gait retraining interventions. With the advent of wearable sensors that have been deemed reliable and valid to measure patient gait performance outcomes commercially available elements are utilized to provide gait evaluation not only in a mobile fashion but also in a less expensive manner. This could also have important implications towards further clinical usage as clinicians could benefit from further knowledge beyond qualitatively assessing gait when a patient returns between visits.

This work is innovative in its use of current positive findings of real time visual feedback by providing the visual real time feedback directly from the patient's prosthetic limb. This is displayed on smart glasses creating a mobile environment in which the training can occur with novel feedback from the integrated sensor. The integrated F/T sensor being utilized has been found valid for the measurement of joint forces and moments. This, in combination with mobile assessment of kinematic and temporal-spatial gait outcomes completes a novel way of training and assessing improvements in dysfunctional gait kinematics. It has been demonstrated that amputees have greater difficulty on non-level surfaces. Truly mobile gait retraining, allowing for real time visual feedback while walking outside of the clinic, has not before been tested.

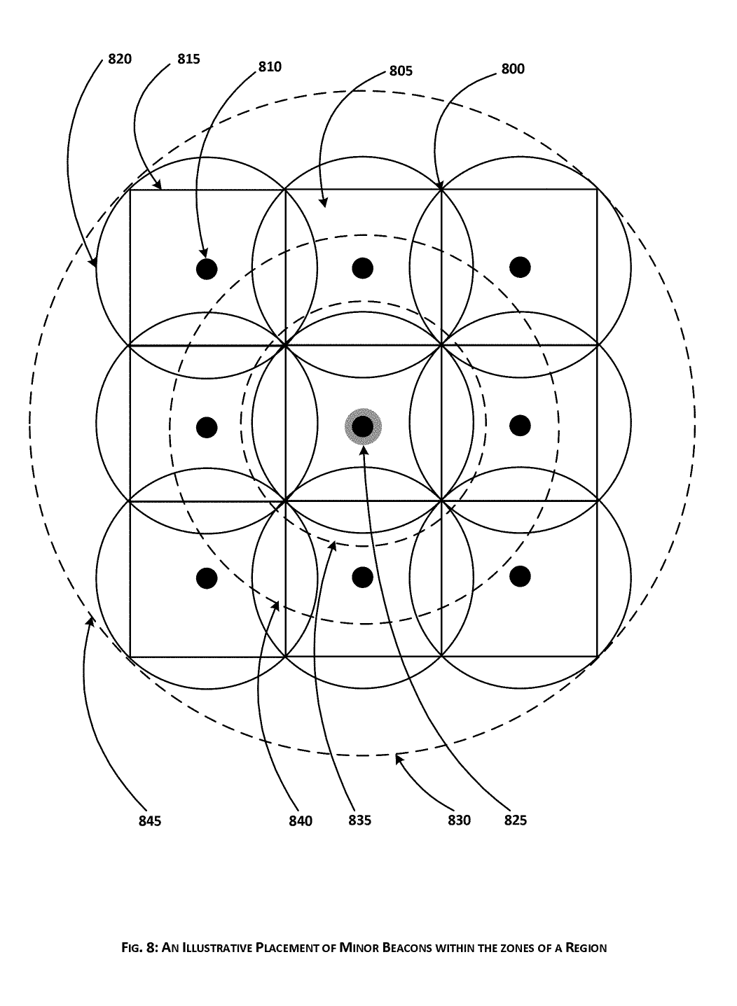

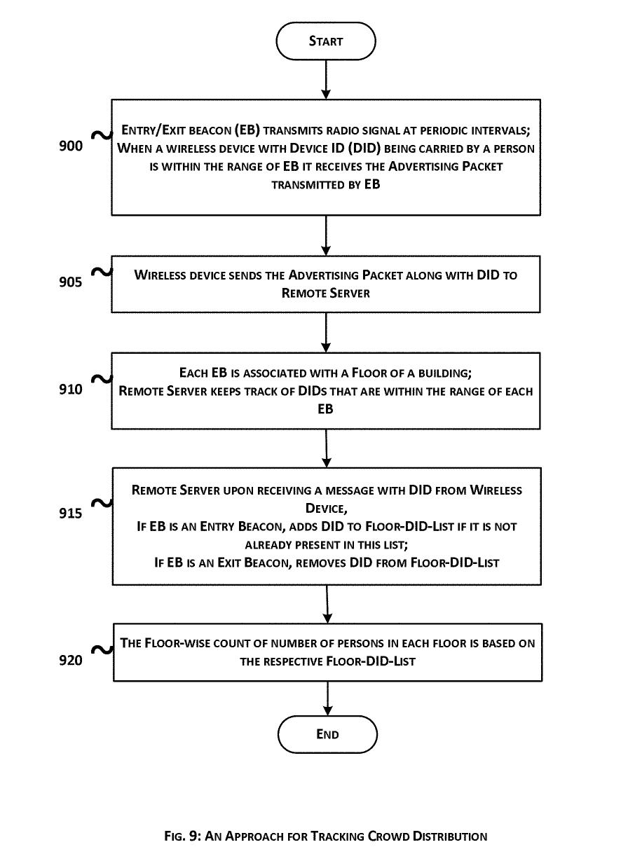

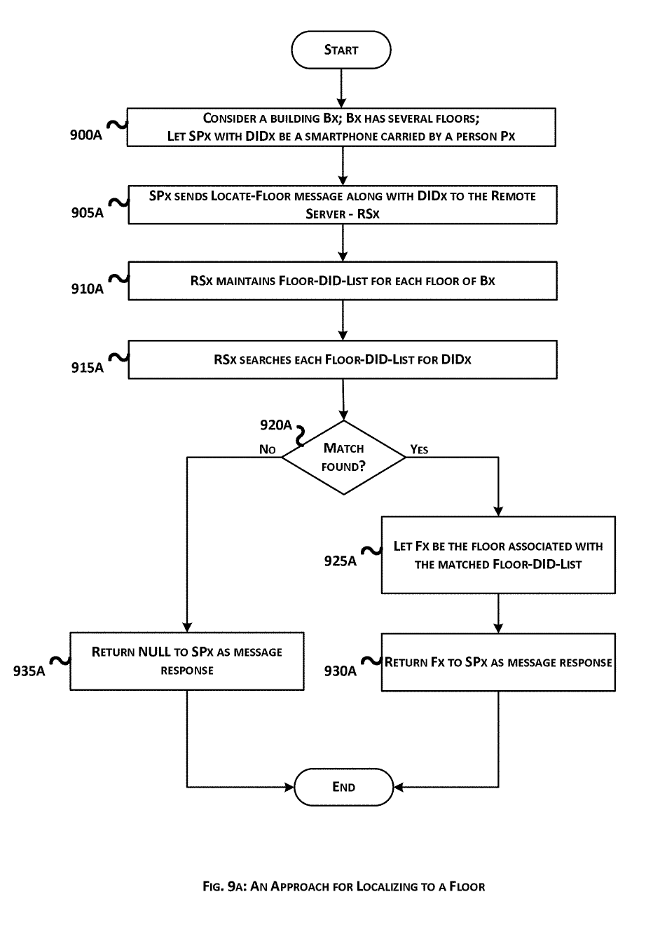

For initial testing, the patient ambulates with the integrated i-Pecs™ sensor (RTC Electronics, Dexter, Mich.) (see, e.g., sensor 58 of FIG. 2) which is programmed to communicate directly with smart glasses via Bluetooth to provide the RTMVF of vertical ground reaction force (VGRF) feedback to the patient as they walk (FIG. 8). The i-Pecs™ has been demonstrated as valid for ground reactive force and joint moment data collection data in this population. Amputees have been demonstrated to have decreased VGRF through the involved limb which has been demonstrated to relate to decreased stance time. Therefore, we will initially utilize VGRF data as the display and ask the patient to match their curve with the normal curve (see, e.g., FIG. 8). Participants will undergo gait retraining. Upon initial evaluation, a standardized checklist of gait deviations will be utilized to review at each subsequent session. These then will remain the standardized deviations for that patient for 8 visits, and the same cues will be used, per deviation, across patients. These cues will be used to associate their gait changes to the feedback changes they are seeing on the display of their smart glasses. Each session will include 1 hour, twice a week for 4 weeks and will include RTMVF training and gait focused physical therapy. It will be randomized how each patient starts their 1-hour session. A faded feedback protocol will be utilized over the last four session to help internalize motor learning. An external focus directs the attention of the learner on the effects of their movements (different walking strategies changing the force curves they seen on the screen versus focusing on their own extremity alignments) and reduces their attentional demands.

While several examples and embodiments of the sensor device, movement analysis and training system, and processes for providing real-time gait training based on sensed movement data are shown in the accompanying figures and described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

### EXAMPLE 2

Gait dysfunction is an impairment that can effect multiple patient populations, both neurologic and orthopedic, including those with limb loss, and become chronic and linger for years. It has been frequently reported that gait retraining with augmented sensory feedback improves dysfunctional lower extremity impairments and related gait patterns including those of amputees. However, a primary criticism of these previous studies is due to the expense and tightly controlled laboratory conditions, conducted with instrumented treadmills and optical systems, many of the findings have had difficulty being applied to realistic clinical environments. Progressing from this previous limitation, a system is provided based on an integrated load cell sensor to provide real time mobile visual feedback (RTMVF) to trans-tibial amputees for gait training.

Translating current positive findings of real time visual feedback into a clinical application was accomplished by providing the visual real time feedback directly from the patient's limb displayed on smart glasses, with the objective of creating a mobile more realistic environment in which the training can occur. The system included a prosthesis-integrated load cell (ipecs, RCT Electronics, Dexter, Mich.) and a variety of commercially available wearable head-up displays. After testing several models, including the Google glass system (Google, Mountain View, Calif.) and the Recon jet (recon instruments, Vancouver, BC), we implemented Vuzix M100 smart glasses (Vuzix, West Henrietta, N.Y.) in the prototype (see FIG. 9).

Establishing connectivity between the different components is an ongoing process. The current prototype uses cable connections between the load cell, a laptop computer, and the smart glasses. Given that all those components have Bluetooth capabilities; an updated wireless prototype is anticipated for future iterations of the development project.

The initial prototype (FIG. 9) allows the capturing, processing and displaying of load cell gait data in close to real time. Initial feasibility tests suggest that a patient can be fitted with the system in about 30 minutes, most of which time is required for the installation of the load cell into the prosthesis structure. The prototype is anticipated to provide the groundwork for subsequent work to determine how to best convert the raw data to the visual warning signal resulting in an RTMVF gait training system for trans-tibial amputees.

Long term goals are the effective supplementation of traditional therapist-based gait retraining with a wearable “assistant” that provides comparable feedback on a patient's gait deviations. This should help improve outcomes for patients who have limited access to specialized health care, and who are, therefore, at risk of adapting habitual gait deviations following limb loss and prosthesis provision.

### EXAMPLE 3

### Methods

The RTMVF system was developed utilizing commercially available and/or previously validated componentry, essentially as described in FIG. 9. Gait data source is a prosthesis-integrated load cell (i-Pecs, RCT Electronics, Dexter, Mich.) capable of measuring precisely kinetic gait variables in lower limb prostheses. The device is semi-permanently installed as part of the load-bearing structure of the limb prosthesis connecting to the rest of the device using standard adapters. Ground reaction forces and moments of force data can be collected at up to 850 Hz and transferred wirelessly or by cable connection to a laptop computer for further processing. In order to provide visual feedback to the patient, a wearable head-up display (M300, Vuzix, West Henrietta, N.Y.) was used. These “smart glasses” contain, positioned at the fringe of the user's normal field of view, a small sized display, the contents of which are retrieved from the computer via Wi-Fi or Bluetooth connection. The display has a resolution that is comparable with small computer screens, yet its position and intended purpose in our context advises against the conveyance of very complex visual information.

Connectivity between the different components is currently realized using cable connection between load cell and laptop computer, and Bluetooth to the smart glasses. In this configuration, the lightweight computer is carried in a pouch on a waist belt by the user.

Feasibility of the system was evaluated using the feedback variable “Stance/step time ratio”, (that is, the duration of a step's stance phase (from initial ground contact to toe-off) in relation to the overall step's duration measured from one initial ground contact to the next initial ground contact on the same side). This parameter correlates with some typical gait deviations in lower limb prosthesis users, and it lends itself to easy capturing by lower-cost, prosthesis-independent sensor equipment for potential translation into the clinic and/or adaptation for different patient populations. Stance and swing components of step cycles were derived by an algorithm that analysed various components of the axial force curve (the sensor's Fz is roughly equivalent to vertical ground reaction force in an external coordinate system, Fiedler G, et al. Criterion and Construct Validity of Prosthesis-Integrated Measurement of Joint Moment Data in Persons With Trans-Tibial Amputation. Journal of Applied Biomechanics. 2014; 30(3):431-438) to determine the appropriate crossings of a 15N and 100N threshold. Examining the sensor data graphically and quantitatively, different strategies were tested to harden the algorithm to outliers and measuring artefacts (Kutina K, Fiedler G. The Use of an Integrated Load Cell as a Mobile Gait Analysis System to detect Gait Events in People with Limb Loss. Paper presented at: ISPO 16th World Congress 2017; Cape Town, South Africa). Timing parameters were established to help the algorithm detect transition steps and turns as non-representative steps for gait analysis and feedback purposes.

A target window of stance/step ratio was established between 0.59 and 0.63, resulting in three discrete output states: too short stance phase (below 59% of step cycle), desirable stance phase (59-63%), and too long stance phase (above 63%). The three states were represented by different feedback colors, displaying a red (for too short stance phase), green (desirable stance phase), or yellow (too long stance phase) screen to the user. The most accurate calculation of stance phase duration with respect to the total step cycle requires the entire step cycle in question to be timed. This makes the feedback information available only after a given analysed step is completed. Accounting for this inevitable latency, the validity of the system in generating feedback variables was investigated in a small sample of steps.

Inclusion criteria for this test was use of a trans-tibial prosthesis for ambulation, absence of acute or chronic health conditions that would affect prosthesis use, and ability to walk without aids for at least 30 minutes. Demographic data and mobility score (PLUS-M¹⁹) were recorded. The test participant was equipped with the RTMVF system and a waist-worn “mobile gait lab” (G-Walk, BTS Engineering, Milan, ITA), and was asked to traverse in self-selected walking speeds repeatedly across a 30-m level walkway. Step phase durations were extracted from the G-Walk data to serve as the validation standard for the respective variables computed from the prosthesis sensor data by our algorithm. Gait symmetry index, a proprietary variable output by the G-Walk software, was recorded as well and correlated to the i-Pecs derived stance/step ratio in order to investigate its appropriateness as feedback variable for gait training. The variable is a composite index that is based on acceleration and gyroscope data through the step cycle. An index of 100 signifies perfect symmetry between the left and the right step with respect to ground contact forces, trunk tilt, and temporal parameters. Bivariate correlation analysis was conducted using IBM SPSS Statistics (Version 24).

### Results

The participant was a 61-year-old female, weighing 58.5 kg and 1.49 m tall, who had been using trans-tibial prostheses for twelve years and had a PLUS-M score at the 79^{th} percentile.

A total of 67 steps were analyzed. Correlation between RTMVF step ratio data and reference data was strong, with a Pearson correlation coefficient of R=0.813 (p<0.001).

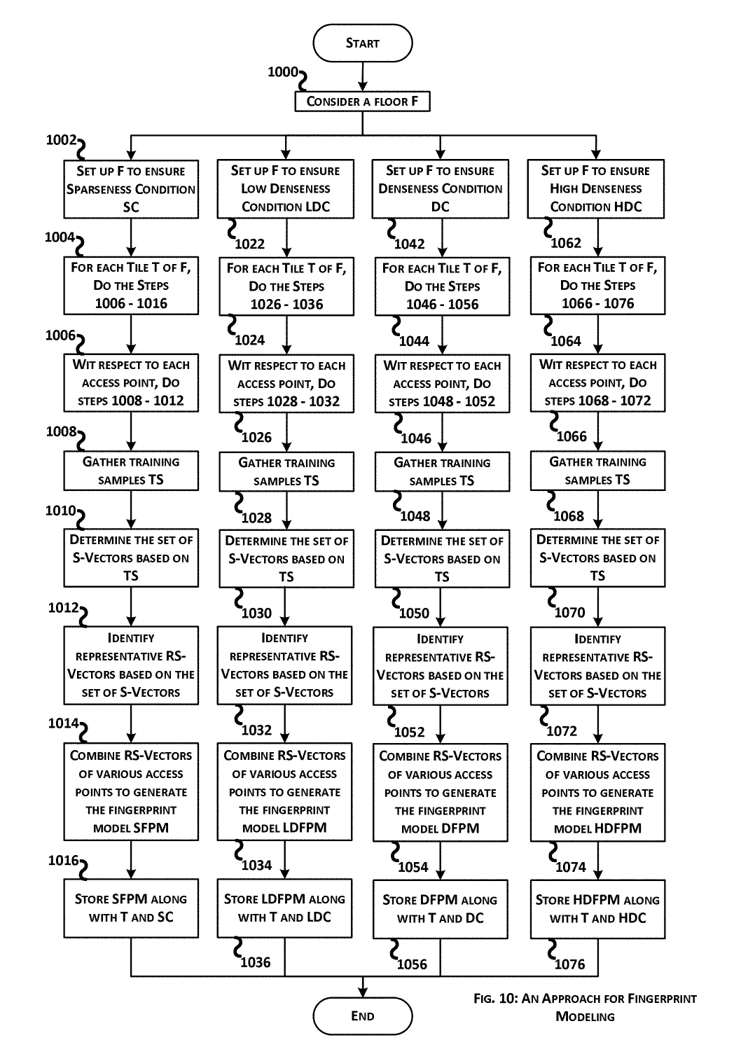

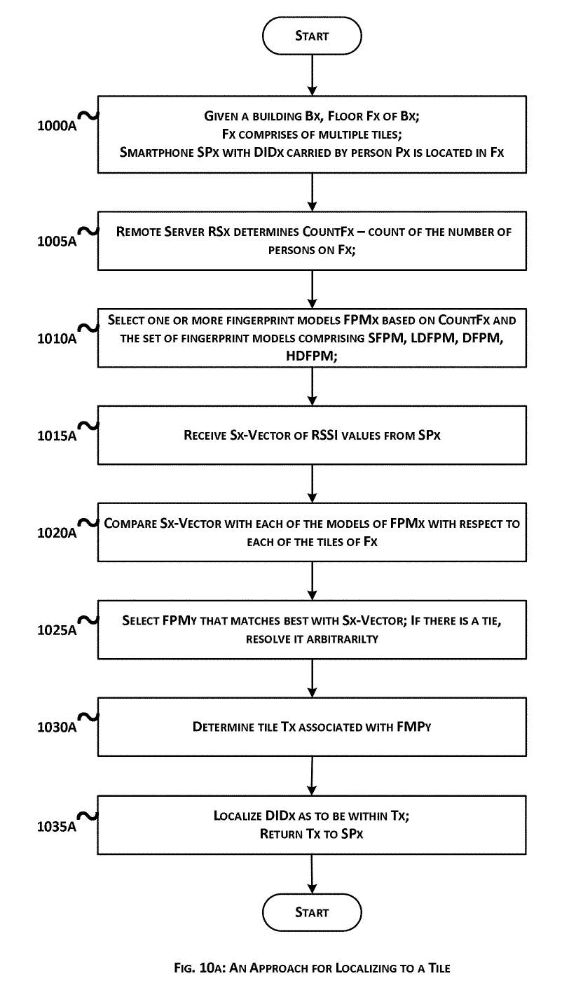

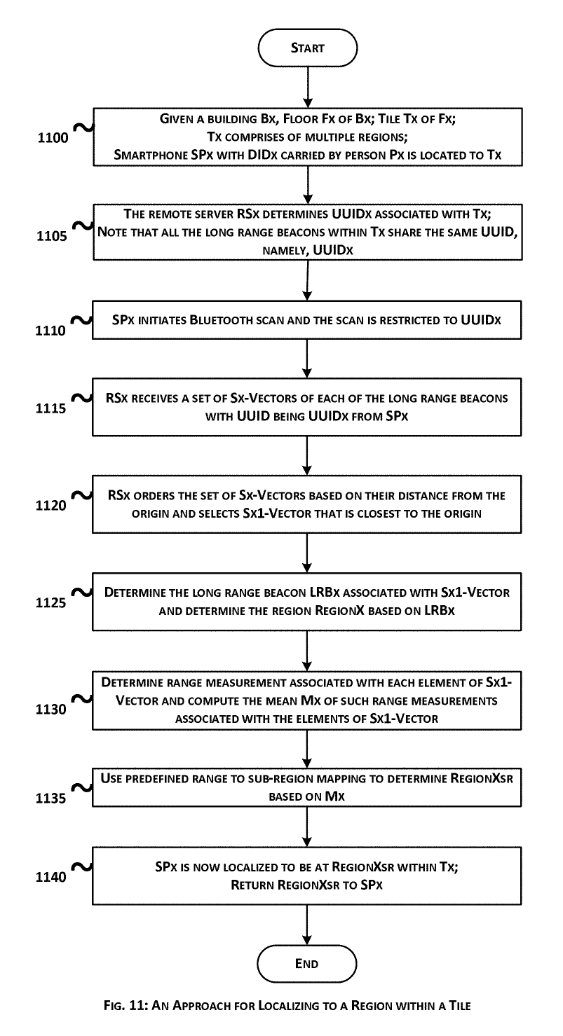

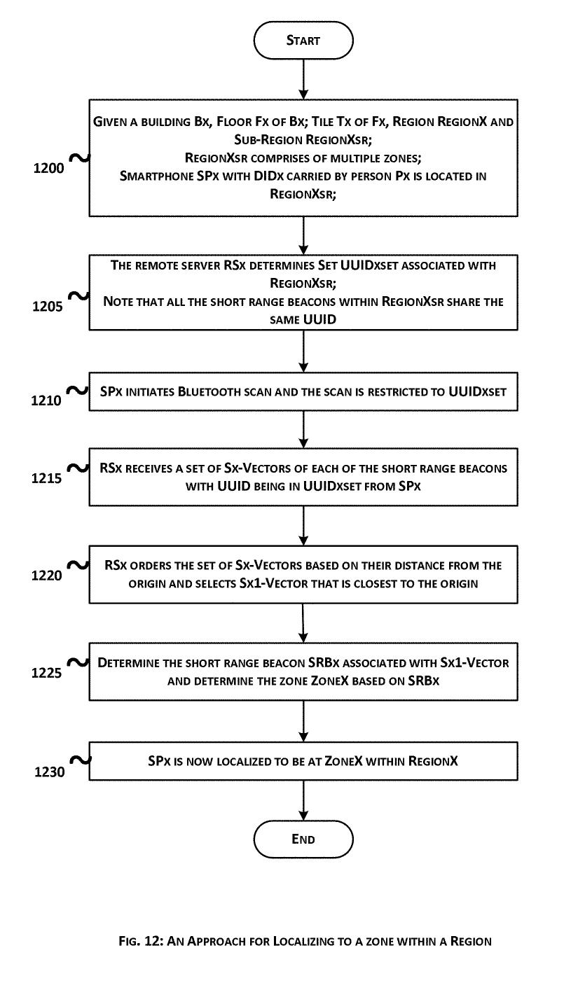

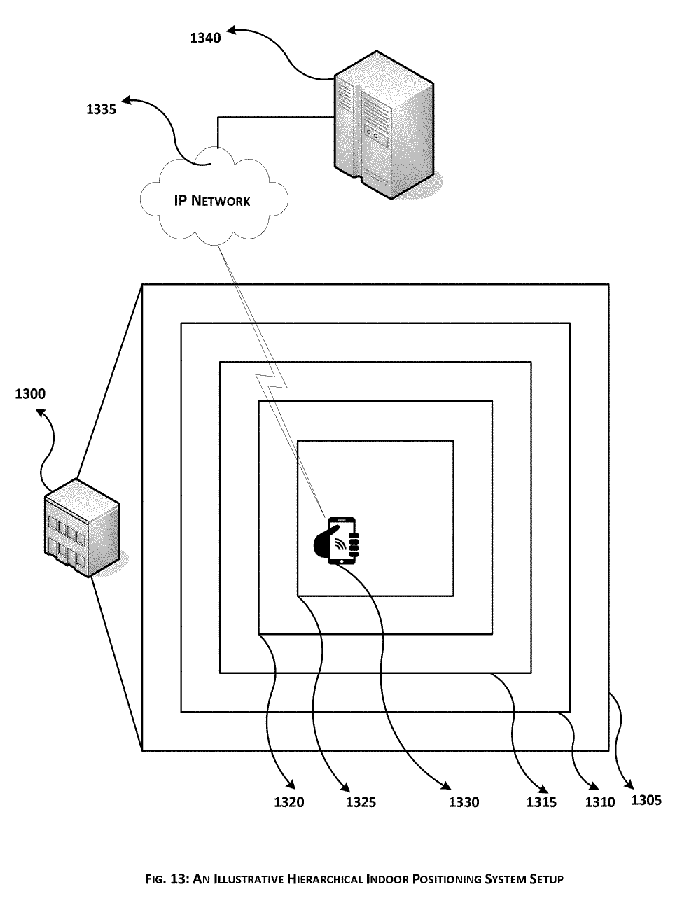

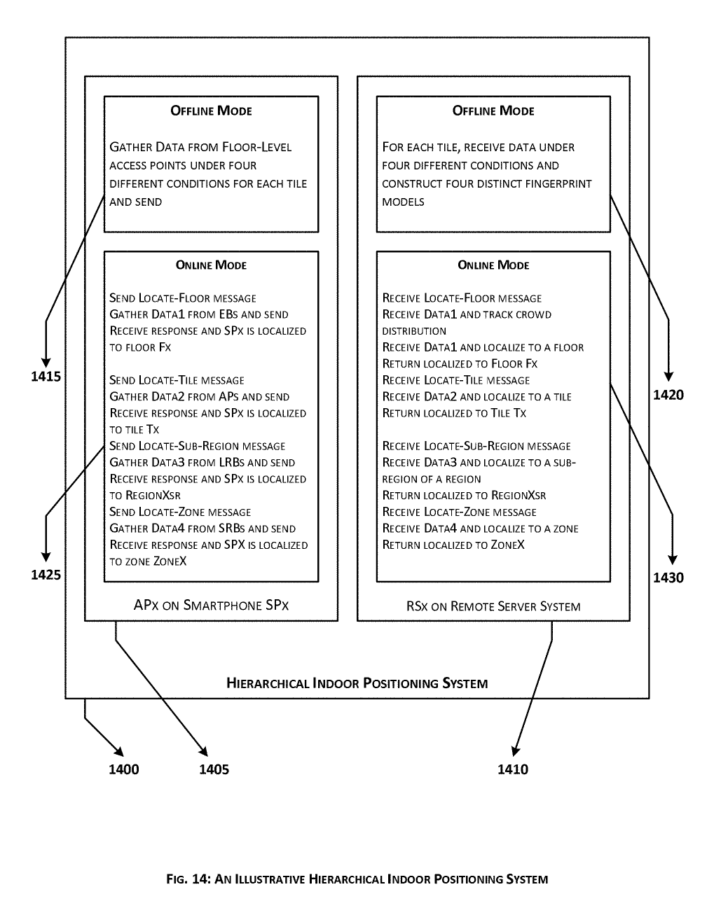

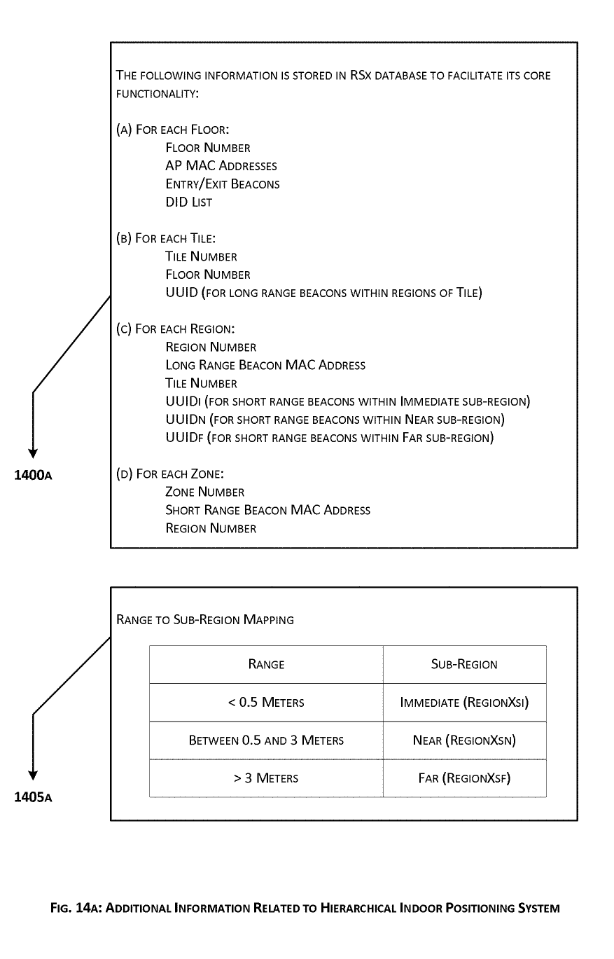

Correlation between variables stance/step ratio and overall gait symmetry index (FIG. 10) across eight data collection sessions was strong as well (R=0.735), indicating that the feedback variable is a good proxy for the primary outcome of interest.

Latency of feedback was less than 1 second and was not perceived as problematic by the test subject. The test suggests that a patient can be fitted with the system in about 30 minutes, most of which time is required for the installation of the load cell into the prosthesis structure.

Findings suggest that our system measures step cycle components with sufficient validity. None of the analyzed steps were classified improperly, and deviations between the two utilized data capture systems did on average not exceed 66 milliseconds or 10.2 percent, a discrepancy that can be deemed acceptable, and may be attributable to the difference in generating kinetics data based on accelerometry (G-Walk) and strain gages (i-Pecs). This leaves the slight delay in displaying the feedback information that is owed to the processing of load cell gait data, as potentially the most relevant difference to treadmill based feedback systems for gait training. Whether the mobile feedback may still be considered “quasi real-time”, and may thus allow the assumption that the function mechanism of the tested methodology is in principle comparable to more conventional approaches, should be tested in a larger scale study.

Our pilot data collection illustrated the advantages of providing real time visual feedback with a mobile system, in terms of efficiency, clinical applicability, and representativeness of data. Once the short preparations, involving attachment and calibration of the equipment, were concluded, collecting gait data on a substantial number of steps required not more time than the participant spent taking those steps. One person was able to administer the test session, as the patient was able to walk safely and in her regular fashion without being notably encumbered by the wearable equipment. The environment in which the training and data collection can occur is very realistic, as the system can be used on most any indoor and outdoor walking surface, including slopes, stairs, and uneven terrains. Even though only one simple variable was extracted and analyzed for the current study, more of the sensor's raw data (3-axial forces and moments) may prospectively be harvested to refine the detection of gait deviations and to inform better feedback displayed to the patient.

Our single subject pilot study did not allow investigating which modifications to the algorithm may be needed on an individual basis. It may be assumed that other users require slightly different feedback information, depending on the severity of their gait deviation and their ability to make the prescribed corrections. Such users may, for instance, benefit from adjustments to the size and location of the “target window” for the proper stance/step ratio.

Findings of the current study support the goal of effectively supplementing traditional therapist-based gait retraining. By expanding patients' exposure to gait therapy interventions beyond the limited sessions with their therapist, training effects should onset swifter and should be better sustainable. In conclusion, a mobile gait analysis and feedback system holds promise for enhanced gait retraining approaches in people with lower limb loss, training or retraining of people with gait abnormalities, and training and rehabilitation of athletes.

The following numbered clauses describe various aspects of the invention:

1. A gait analysis, training, and retraining system comprising:

a sensor configured to measure one or more attributes of gait of a patient; and
a controller in communication with a sensory output device, the controller configured to, repeatedly, monitor a patient's gait in real-time and to provide real-time feedback to the patient: receive and process information from the sensor representative of one or more attributes of the gait of the patient;
generate a data set corresponding to the information from the sensor representative of one or more attributes of the gait of the patient;
compare the generated data set to reference data indicating optimal values for a data set corresponding to the information from the sensor representative of one or more attributes of the gait of a patient; and
cause the display to provide feedback comprising gait analysis based, at least in part, on the comparison between the generated data set and the reference data and at least indicating in the feedback if the generated data set is within defined tolerances relative to the reference data.

2. The system of clause 1, wherein the sensory output device is a wearable display, such as a wearable smart display device or head up display (HUD).

3. The system of clause 1, wherein the sensory output device is a sound transducer, such as a headphone, or a haptic device, such as a vibration motor.

4. The system of clause 1, wherein the sensor is a force, or a force and torque (F/T sensor) configured to measure forces and torque applied to a leg of a patient.

5. The system of clause 4, wherein the F/T sensor is a six-axis F/T sensor.

6. The system of clause 4, wherein the force sensor measures at least Fz (force applied in a superior direction toward the head of a patient).

7. The system of clause 4, wherein the F/T sensor is provided in a leg prosthesis, such as a trans-tibial, or a trans-femoral prosthesis.

8. The system of clause 4, wherein the F/T sensor is configured to be wearable by a patient.

9. The system of clause 8, wherein the wearable F/T sensor is provided in a shoe, a shoe insert, a shoe outsole, or a shoe attachment.

10. The system of clause 1, wherein the sensor is a wearable inertial measurement unit configured to be worn by the patient comprising at least one accelerometer and/or at least one gyroscope.

11. The system of clause 10, wherein the inertial measurement unit comprises three orthogonally positioned accelerometers for measuring acceleration along the x, y, and z axes and three gyroscopes oriented along the x, y, and z axes respectively.

12. The system of any one of clauses 1-11, wherein the sensory output device is a display providing a binary signal comprising a first visual signal when the generated data set is within defined tolerances relative to the reference data and a second visual signal different from the first visual signal when the generated data set is not within defined tolerances relative to the reference data.

13. The system of clause 12, wherein the display provides a color signal indicating when the generated data set is not within defined tolerances relative to the reference data.

14. The system of clause 1, wherein the controller and the sensory output device communicate wirelessly.

15. The system of any one of clauses 1-14, wherein the reference data is obtained from:

a patient performing the one or more physical actions in an optimal, desirable, or clinically acceptable manner, for the one or more physical actions; and/or
one or more other patients, including statistical data, algorithms, or other values derived from data obtained from other patients.

16. A method of analyzing and/or training gait in a patient, the method comprising, using a computer-implemented process, repeatedly:

receiving and processing, in a computer, information from a sensor on the patient configured to measure one or more attributes of gait of a patient representative of one or more attributes of the gait of the patient during one or more physical actions relating to gait, performed by the patient;
generating, in the computer, a data set corresponding to the information from the sensor representative of one or more attributes of the gait of the patient;
comparing, in the computer, the generated data set to reference data indicating optimal values for a data set corresponding to the information from the sensor representative of one or more attributes of the gait of a patient; and
generating, with the computer, an output causing a sensory output device to provide feedback comprising gait analysis based, at least in part, on the comparison between the generated data set and the reference data and at least indicating in the feedback if the generated data set is within defined tolerances relative to the reference data.

17. The method of clause 16, wherein the sensory output device is a wearable display device.

18. The method of clause 16 or 17, wherein the patient has a lower extremity amputation having a leg prosthesis, such as a trans-tibial, or a trans-femoral prosthesis, and the sensor is integrated into the prosthesis.

19. The method of clause 16 or 17, wherein the patient is missing a leg or a portion thereof below the hip, femur, knee, or ankle.

20. The method of clause 16 or 17, wherein the patient has a gait imbalance, such as from injury, surgery, congenital defect, disease or condition, such as, without limitation, from an ankle, leg, hip, or spine injury, multiple sclerosis, osteoarthritis, cerebral palsy, spinal cord injury, post-polio syndrome, post-stroke conditions, old age.

21. The method of any one of clauses 16-20, wherein the sensor is a force and torque (F/T sensor) configured to measure forces and torque applied to a leg of a patient.

22. The method of clause 21, wherein the F/T sensor is a six-axis F/T sensor.

23. The method of clause 21, wherein the force sensor measures Fz (force applied in a superior direction toward the head of a patient).

24. The method of clause 21, wherein the F/T sensor is provided in a leg prosthesis, such as a trans-tibial, or a trans-femoral prosthesis.

25. The method of clause 21, wherein the F/T sensor is provided in a shoe, a shoe insert, a shoe outsole, or a shoe attachment.

26. The method of any one of clauses 16-25, wherein the sensor is a wearable inertial measurement unit configured to be worn by the patient comprising at least one accelerometer and/or at least one gyroscope, e.g., wherein the inertial measurement unit comprises three orthogonally positioned accelerometers for measuring acceleration along the x, y, and z axes and three gyroscopes oriented along the x, y, and z axes respectively.

27. The method of any one of clauses 17-26, wherein the display provides a first visual signal when the generated data set is within defined tolerances relative to the reference data and a second visual signal different from the first visual signal when the generated data set is not within defined tolerances relative to the reference data.

28. The method of any one of clauses 17-27, wherein the display provides a color signal indicating when the generated data set is not within defined tolerances relative to the reference data.

29. The method of any one of clauses 17-28, wherein the controller and the display communicate wirelessly.

30. The method of any one of clauses 12-25, wherein the reference data is obtained from:

the patient when performing the one or more physical actions in an optimal, desirable, or clinically acceptable manner, for the one or more physical actions; and/or
one or more other patients or sources, including statistical data, algorithms, or other values derived from data obtained from other patients or sources.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

What is claimed is:

1. A gait analysis, training, and retraining system comprising:
a sensor configured to measure one or more attributes of gait of a patient; and
a controller in communication with a sensory output device, the controller configured to, repeatedly, monitor a patient's gait in real-time and to provide real-time feedback to the patient:
receive and process information from the sensor representative of one or more attributes of the gait of the patient;
generate a data set corresponding to the information from the sensor representative of one or more attributes of the gait of the patient;
compare the generated data set to reference data indicating optimal values for a data set corresponding to the information from the sensor representative of one or more attributes of the gait of a patient; and
cause the display to provide feedback comprising gait analysis based, at least in part, on the comparison between the generated data set and the reference data and at least indicating in the feedback if the generated data set is within defined tolerances relative to the reference data,
wherein the controller and the sensory output device optionally communicate wirelessly.

2. The system of claim 1, wherein the sensory output device is a wearable display, such as a wearable smart display device or head up display (HUD) or a sound transducer, such as a headphone, or a haptic device, such as a vibration motor.

3. The system of claim 1, wherein the sensor is a force, or a force and torque (F/T sensor) configured to measure forces and torque applied to a leg of a patient, such as a six-axis F/T sensor.

4. The system of claim 3, wherein the force sensor measures at least Fz.

5. The system of claim 3, wherein the F/T sensor is provided in a leg prosthesis, such as a trans-tibial, or a trans-femoral prosthesis.

6. The system of claim 3, wherein the F/T sensor is configured to be wearable by a patient, such as in a shoe, a shoe insert, a shoe outsole, or a shoe attachment.

7. The system of claim 1, wherein the sensor is a wearable inertial measurement unit configured to be worn by the patient comprising at least one accelerometer and/or at least one gyroscope, such as three orthogonally positioned accelerometers for measuring acceleration along the x, y, and z axes and three gyroscopes oriented along the x, y, and z axes respectively.

8. The system of claim 1, wherein the sensory output device is a display providing a binary signal comprising a first visual signal when the generated data set is within defined tolerances relative to the reference data and a second visual signal different from the first visual signal when the generated data set is not within defined tolerances relative to the reference data, optionally the display provides a color signal indicating when the generated data set is not within defined tolerances relative to the reference data.

9. The system of claim 1, wherein the reference data is obtained from:
a patient performing the one or more physical actions in an optimal, desirable, or clinically acceptable manner, for the one or more physical actions; and/or
one or more other patients, including statistical data, algorithms, or other values derived from data obtained from other patients.

10. A method of analyzing and/or training gait in a patient, the method comprising, using a computer-implemented process, repeatedly:
receiving and processing, in a computer, information from a sensor on the patient configured to measure one or more attributes of gait of a patient representative of one or more attributes of the gait of the patient during one or more physical actions relating to gait, performed by the patient;
generating, in the computer, a data set corresponding to the information from the sensor representative of one or more attributes of the gait of the patient;
comparing, in the computer, the generated data set to reference data indicating optimal values for a data set corresponding to the information from the sensor representative of one or more attributes of the gait of a patient; and
generating, with the computer, an output causing a sensory output device to provide feedback comprising gait analysis based, at least in part, on the comparison between the generated data set and the reference data and at least indicating in the feedback if the generated data set is within defined tolerances relative to the reference data,
wherein the controller and the display optionally communicate wirelessly.

11. The method of claim 10, wherein the sensory output device is a wearable display device.

12. The method of claim 10, wherein the patient has a lower extremity amputation having a leg prosthesis, such as a trans-tibial, or a trans-femoral prosthesis, and the sensor is integrated into the prosthesis.

13. The method of claim 10, wherein the patient has a gait imbalance, such as from injury, surgery, congenital defect, disease or condition, such as, without limitation, from an ankle, leg, hip, or spine injury, multiple sclerosis, osteoarthritis, cerebral palsy, spinal cord injury, post-polio syndrome, post-stroke conditions, or old age.

14. The method of claim 10, wherein the sensor is a force and torque (F/T sensor) configured to measure forces and torque applied to a leg of a patient, such as a six-axis F/T sensor.

15. The method of claim 14, wherein the force sensor measures Fz.

16. The method of claim 14, wherein the F/T sensor is provided in a leg prosthesis, such as a trans-tibial, or a trans-femoral prosthesis.

17. The method of claim 14, wherein the F/T sensor is provided in a shoe, a shoe insert, a shoe outsole, or a shoe attachment.

18. The method of claim 10, wherein the sensor is a wearable inertial measurement unit configured to be worn by the patient comprising at least one accelerometer and/or at least one gyroscope, e.g., wherein the inertial measurement unit comprises three orthogonally positioned accelerometers for measuring acceleration along the x, y, and z axes and three gyroscopes oriented along the x, y, and z axes respectively.

19. The method of claim 10, wherein the display provides a first visual signal when the generated data set is within defined tolerances relative to the reference data and a second visual signal different from the first visual signal when the generated data set is not within defined tolerances relative to the reference data, wherein the display optionally provides a color signal indicating when the generated data set is not within defined tolerances relative to the reference data.

20. The method of claim 10, wherein the reference data is obtained from:
the patient when performing the one or more physical actions in an optimal, desirable, or clinically acceptable manner, for the one or more physical actions; and/or
one or more other patients or sources, including statistical data, algorithms, or other values derived from data obtained from other patients or sources.
